(19) United States
(12) Patent Application Publication
(10) Pub. No.: US 2019/0022654 A1
(43) Pub. Date: Jan. 24, 2019

(54) METHOD FOR INCUBATING LIQUIDS

(71) Applicants: Baxalta Incorporated, Bannockburn, IL (US); Baxalta GmbH, Zug (CH)

(72) Inventors: Nikolaus HAMMERSCHMIDT, Vienna (AT); Jure SENCAR, Vienna (AT); Alois JUNGBAUER, Vienna (AT); Duarte Lima MARTINS, Vienna (AT)

(73) Assignees: Baxalta Incorporated, Bannockburn, IL (US); Baxalta GmbH, Zug (CH)

(21) Appl. No.: 16/031,171

(22) Filed: Jul. 10, 2018

(30) Foreign Application Priority Data
Jul. 10, 2017 (EP) 17180477.6
Jan. 30, 2018 (EP) 18154196.2

(51) Int. Cl. B01L 3/00; A61P 31/14; B01F 13/00; C12N 7/02; C12N 7/06

(52) U.S. Cl. CPC B01L 3/502761; A61P 31/14; B01F 13/0059; C12N 2760/00063; C12N 7/02; C12N 7/06; B01L 3/502769

(57) ABSTRACT

The present invention relates a method for incubating liquids, to a method for preparing a biopharmaceutical drug, and to a device for the preparation of a biopharmaceutical drug.

### CROSS-REFERENCE TO RELATED APPLICATIONS

This application claims priority from European Patent Application No. EP 17180477.6, filed Jul. 10, 2017 and European Patent Application No. 18154196.2, filed Jan. 30, 2018, which are hereby incorporated by reference in their entirety.

### FIELD OF THE INVENTION

The present invention relates to a method for incubating liquids, to a method for preparing a biopharmaceutical drug, and to a device for the preparation of a biopharmaceutical drug.

### BACKGROUND

In many continuously operating processes, liquids are mixed and then incubated by passing them through processing equipment such as tubes or columns. However, when passing through a structure that is used in the process, parts of the liquid that are closer to the surface of the structure tend to flow at a lower velocity than parts of the liquid that are more distant from the surface of the structure. For example, when a liquid mixture flows through a hollow tube, parts of the liquid in the center of the tube tend to flow at a higher velocity than parts of the liquid in the periphery. As a result, different parts of the liquid have different residence times, even when all parts of the liquid enter the structure at the same time. In other words, the different parts of the liquid show a distribution of residence times. If there is a big difference in flow through time between the different parts of the liquid, the residence time distribution is broad; if there is a small difference in flow through time between the different parts of the liquid, the residence time distribution is narrow.

A narrow residence time distribution is advantageous when liquid mixtures are to be incubated for a defined period of time. For example, in a continuous biopharmaceutical production process, continuous virus inactivation can be achieved by mixing a biopharmaceutical-containing liquid with virus-inactivating agents and incubating the mixture by passing it through a structure used in the process for a defined time period. A narrow residence time distribution allows that all parts of the liquid of the mixture are incubated with the virus inactivating agent for a similar, i.e. the desired period of time. That way, it could be avoided that some parts of the liquid are exposed to the virus-inactivating agent for too long, which could harm the biopharmaceutical drug, whereas other parts of the liquid are not exposed to the virus-inactivating agent for long enough, which could lead to incomplete virus inactivation.

The currently known methods for incubating flowing liquids do not provide for a narrow residence time distribution, or they suffer from other severe shortcomings. For example, the simplest approach for incubating a liquid mixture in a continuous production process would be to pass it through a hollow tube that is sufficiently long to provide the desired minimum residence time. However, the residence time distribution in a hollow tube is extremely broad and irreproducible. Static mixers could be added into the tube to promote radial mixing (Ref. 1). However, for such setup scale-up can be an issue as well as the fitting of static mixers into long stretches of tube.

Alternatively, so-called coiled flow inverters (CFI) work by passing a liquid mixture through a coiled tube that has additional 90° bends (Ref. 2). This setup is supposed to increase radial mixing while minimizing axial mixing, thereby narrowing the residence distribution. The system has been described recently for the use in continuous virus inactivation (Refs. 3, 4), and the same setup has recently been used to narrow the residence time distribution in an impurity precipitation step (Ref. 5). However, the CFI has been proven to work only with tube diameters of 2-3 mm, and scale-up remains challenging because fluid dynamics in the system change with tube dimensions. The CFI is also limited to a single flow rate for each given design.

Recently, segmenting a product stream in a microreactor by introducing an immiscible separation medium has been suggested for continuous virus inactivation with narrow residence time distribution (Ref. 6). However, such method is limited to the use of microreactors, which renders scale-up very difficult.

Due to the above-described lack of suitable methods for incubating flowing liquids with a narrow residence time distribution, currently time-sensitive incubation is often performed in batch mode rather than continuous mode. In batch mode, the liquid mixture is incubated in a container while the flow is interrupted for the time of incubation. As a result, productivity (e.g. in terms of the amount of incubated liquid per period of time, or in terms of the amount of a biopharmaceutical drug produced per period of time) in batch mode is generally lower than productivity in continuous mode.

In view of the above, there is great demand for improved methods that that allow to incubate liquids over a defined period of time, while providing a high productivity.

### DESCRIPTION OF THE INVENTION

The present invention meets the above-described needs and solves the above-mentioned problems in the art by providing the embodiments described below:

In particular, the present inventors have surprisingly found that passing a liquid through a structure having multiple interconnected channels provides for a narrower residence time distribution than previously known methods. Thus, according to the invention, a mixture of at least two liquids can be incubated by mixing said at least two liquids, and passing the mixture through a structure having multiple interconnected channels, wherein the mixing and passing is carried out continuously.

The inventors have also found that the structure having multiple interconnected channels can be a packed bed of non-porous beads. In this embodiment, the interconnected channels are formed by the spaces between the non-porous beads. The inventors then performed numerous experiments to find out which properties affect the residence time distribution. The inventors surprisingly found that the mean particle diameter and particle size distribution of the beads forming the packed bed have the highest impact on residence time distribution in the tested range. Specifically, the inventors found that said packed bed of non-porous beads provides for a particularly narrow residence time distribution when the beads have a mean particle diameter in the range of 0.05 mm to 1 mm, and when the particle size distribution is narrow. Moreover, the inventors have found that larger volumes of the packed beds of non-porous beads result in narrower residence time distributions. Further, according to the invention, longer beds of beads (e.g. in forms of columns) also result in narrower residence time distributions.

In contrast to many currently used methods, the methods of the present invention can be scaled-up easily. This is because the method of the present invention is not very sensitive to changes in flow rates and superficial linear velocities, and because the residence time distribution gets narrower when using packed beds of non-porous beads that have larger volumes and are longer. Thus, the method of the present invention can easily be integrated into commercial production processes.

Overall, the present invention provides improved means for incubating liquids by providing the preferred embodiments described below:
1. A method for incubating a mixture of at least two liquids, the method comprising:
   i) mixing said at least two liquids to obtain a mixture; and
   ii) passing said mixture through a structure having multiple interconnected channels, thereby incubating said mixture.
2. The method according to item 1, wherein the method is a continuous-flow method.
3. The method according to item 1 or 2, wherein said mixing and passing is carried out continuously.
4. The method according to any one of the preceding items, wherein the structure having multiple interconnected channels is a packed bed of non-porous beads.
5. The method according to item 4, wherein the non-porous beads are inert non-porous beads.
6. The method according to item 4 or item 5, wherein the non-porous beads are glass beads, or ceramic beads, or plastic beads such as PMMA beads, or steel beads.
7. The method according to any one of items 4 to 6, wherein the mean particle diameter of the non-porous beads is in the range of 0.05-1 mm, preferably in the range of 0.05-0.6 mm, more preferably 0.05 to 0.5 mm, and most preferably in the range of 0.05-0.3 mm.
8. The method according to any one of items 4 to 7, wherein 95% of the non-porous beads do not deviate from the mean particle diameter by more than 50%, preferably not more than 35%, most preferably not more than 20%.
9. The method according to any one of items 1 to 8, wherein the structure having multiple interconnected channels has a length of at least 5 cm, or at least 10 cm, or at least 20 cm, or at least 30 cm, or at least 50 cm, or at least 70 cm, or at least 100 cm.
10. The method according to any one of items 1 to 9, wherein the structure having multiple interconnected channels has a length of at least 20 cm.
11. The method according to any one of items 4 to 10, wherein the packed bed of non-porous beads is obtainable by a method which comprises subjecting said non-porous beads to a vibration treatment.
12. The method according to any one of items 4 to 11, wherein for the packed bed of non-porous beads, the fraction of the volume of voids over the total volume is in the range of 0.2 to 0.45.
13. The method according to any one of items 4 to 12, wherein for the packed bed of non-porous beads, the fraction of the volume of voids over the total volume is in the range of 0.37 to 0.42.
14. The method according to any one of items 4 to 13, wherein the packed bed of non-porous beads is contained in a column and/or a reactor.
15. The method according to item 14, wherein the column has a diameter of more than 5 mm, preferably a diameter of at least 10 mm.
16. The method according to any one of items 4 to 15, wherein the void volume of the packed bed of non-porous beads is at least 10 mL, preferably at least 40 mL, more preferably at least 150 mL, still more preferably at least 470 mL and still more preferably at least 700 mL.
17. The method according to any one of items 1, 9 and 10, wherein the structure having multiple interconnected channels is a monolith or a precast structure such as a 3D printed geometry.
18. The method of item 17, wherein the structure having multiple interconnected channels is a monolith, and wherein for the monolith, the fraction of the volume of voids over the total volume is in the range of 0.5 to 0.75.
19. The method according to any one of items 1 to 18, wherein the method is for virus inactivation, and wherein a first of said at least two liquids is a liquid potentially containing a virus, and wherein a second liquid of said at least two liquids comprises a virus-inactivating agent.
20. The method according to item 19, wherein said first liquid comprises a biopharmaceutical drug.
21. The method according to item 19 or 20, wherein the method is for virus inactivation of enveloped viruses.
22. The method according to any one of items 19 to 21, wherein said virus is a retrovirus and/or a virus of the Flaviviridae family.
23. The method of item 22, wherein said virus is a retrovirus, preferably X-MuLV.
24. The method of item 22, wherein said virus is a virus of the Flaviviridae family, preferably BVDV.
25. The method according to any one of items 19 to 24, wherein the virus-inactivating agent is a solvent/detergent mixture suitable for solvent/detergent virus-inactivating treatment, or an acidic solution suitable for low pH virus-inactivating treatment.
26. The method according to any one of items 19 to 25, wherein the virus-inactivating agent is a solvent/detergent mixture for solvent-detergent treatment.
27. The method according to any one of items 19 to 26, wherein the method achieves at least a 1 Log10 reduction value (LRV), at least a 2 LRV, at least a 4 LRV or at least a 6 LRV for at least one virus.
28. The method according to item 27, wherein said at least one virus is a virus according to any one of items 22-24.
29. The method according to any one of items 1 to 28, wherein the superficial linear velocity of said mixture through said structure is equal to or lower than 600 cm/h, or equal to or lower than 300 cm/h, or equal to or lower than 200 cm/h, or equal to or lower than 100 cm/h, or equal to or lower than 50 cm/h, or equal to or lower than 20 cm/h.
30. The method according to any one of items 1 to 29, wherein the Bodenstein number of said mixture when passing through said structure having multiple interconnected channels is equal to or higher than 50, preferably equal to or higher than 300, more preferably equal to or higher than 400, still more preferably equal to or higher than 500, still more preferably equal to or higher than 600, most preferably equal to or higher than 800.
31. A method for preparing a biopharmaceutical drug, the method comprising performing the method of any one of items 20 to 31, and recovering said biopharmaceutical drug.
32. A device for the preparation of a biopharmaceutical drug, the device comprising a packed bed of non-porous beads.
33. The device according to item 32, wherein the non-porous beads are inert non-porous beads.
34. The device according to item 32 or item 33, wherein the non-porous beads are glass beads, or ceramic beads, or plastic beads such as PMMA beads, or steel beads.
35. The device according to any one of items 32 to 34, wherein the mean particle diameter of the non-porous beads is in the range of 0.05-1 mm, preferably in the range of 0.05-0.6 mm, more preferably in the range of 0.05-0.5 mm, most preferably in the range of 0.05-0.3 mm.
36. The device according to any one of items 32 to 35, wherein the non-porous beads do not deviate from the mean particle diameter by more than 50%, preferably not more than 35%, most preferably not more than 20%.
37. The device according to any one of items 32 to 36, wherein the packed bed of non-porous beads has a length of at least 5 cm, or at least 10 cm, or at least 20 cm, or at least 30 cm, or at least 50 cm, or at least 70 cm, or at least 100 cm.
38. The device according to any one of items 32 to 37, wherein the packed bed of non-porous beads has a length of at least 20 cm.
39. The device according to any one of items 32 to 38, wherein the packed bed of non-porous beads is obtainable by a method which comprises subjecting said non-porous beads to a vibration treatment.
40. The device according to any one of items 32 to 39, wherein for the packed bed of non-porous beads, the fraction of the volume of voids over the total volume is in the range of 0.2 to 0.45.
41. The device according to any one of items 32 to 39, wherein for the packed bed of non-porous beads, the fraction of the volume of voids over the total volume is in the range of 0.37 to 0.42.
42. The device according to any one of items 32 to 41, wherein the packed bed of non-porous beads is contained in a column and/or a reactor.
43. The device according to item 42, wherein the column has a diameter of more than 5 mm, preferably a diameter of at least 10 mm.
44. The device according to any one of items 32 to 43, wherein the void volume of the packed bed of non-porous beads is at least 10 mL, preferably at least 40 mL, more preferably at least 150 mL, still more preferably at least 470 mL and still more preferably at least 700 mL.
45. The device according to any one of items 32 to 44, wherein the device additionally comprises one or multiple mixers, which are connected to the packed bed of non-porous beads.
46. The device according to item 45, wherein the mixer is a static mixer such as a T-junction mixer, or wherein the mixer is a dynamic mixer such as a dynamic stirrer.
47. The device according to any one of items 32 to 46, wherein the device additionally comprises a filter, and wherein the filter is preferably positioned between the packed bed of non-porous beads and a static mixer according to item 45 or 46.
48. The device according to item 47, wherein the filter has a pore size of 0.2 μm.
49. The device according to any one of items 32 to 48, wherein the device is a continuous-flow reactor.
50. A method for modification of a continuous-flow virus inactivation process, wherein the modification comprises using a structure having multiple interconnected channels for continuous-flow virus inactivation, and passing a mixture of at least two liquids through said structure, thereby incubating said mixture for virus inactivation.
51. The method according to item 50, wherein said continuous-flow virus inactivation process is a process for the preparation of a biopharmaceutical drug.
52. The method according to any one of items 50 to 51, wherein said virus inactivation process uses a virus-inactivating agent for virus inactivation, and wherein a first of said at least two liquids is a liquid potentially containing a virus, and wherein a second liquid of said at least two liquids comprises a virus-inactivating agent.
53. The method according to any one of items 50 to 52, wherein said virus inactivation process is for virus inactivation of enveloped viruses.
54. The method according to item 52 or 53, wherein the virus-inactivating agent used in said virus inactivation process is a solvent/detergent mixture suitable for solvent/detergent virus-inactivating treatment, or an acidic solution suitable for low pH virus-inactivating treatment.
55. The method according to item 54, wherein the virus-inactivating agent used in said virus inactivation process is a solvent/detergent mixture for solvent-detergent treatment.
56. The method according to any one of items 50 to 55, wherein the modification comprises modifying the virus inactivation process to achieve at least a 1 Log10 reduction value (LRV), at least a 2 LRV, at least a 4 LRV or at least a 6 LRV for at least one virus.
57. The method according to any one of items 50 to 56, wherein the modification comprises modifying the virus inactivation process such that Bodenstein number of the mixture passing through said structure having multiple interconnected channels is equal to or higher than 50, preferably equal to or higher than 300, more preferably equal to or higher than 400, still more preferably equal to or higher than 500, still more preferably equal to or higher than 600, most preferably equal to or higher than 800.
58. The method according to any one of items 50 to 57, wherein the modification comprises modifying the virus inactivation process such that the superficial linear velocity of the mixture through said structure is equal to or lower than 600 cm/h, or equal to or lower than 300 cm/h, or equal to or lower than 200 cm/h, or equal to or lower than 100 cm/h, or equal to or lower than 50 cm/h, or equal to or lower than 20 cm/h.
59. The method according to any one of items 50 to 58, wherein the modification comprises using a structure having multiple interconnected channels as defined in any one of items 4-18.
60. The method according to any one of items 56 to 59, wherein the modification comprises adjusting the flow through time of said mixture in said structure to achieve said Log10 reduction value (LRV), and wherein the flow through time is adjusted by adjusting the superficial linear velocity of the mixture and/or the void volume of said structure.

It will be understood that while the above preferred embodiments recite “incubating a mixture of at least two liquids” and “mixing said at least two liquids to obtain a mixture”, the invention is not limited to the use of at least two liquids. For example, a method of the invention can also be a method for incubating a mixture of at least one liquid and at least one solid, the method comprising i) mixing said at least one liquid and said at least one solid to obtain a mixture; and ii) passing said mixture through a structure having multiple interconnected channels, thereby incubating said mixture. For example, in a method for virus inactivation according to the invention, a virus-inactivating agent may be added in form of at least one solid. Preferably, the solid can be in form of a powder. It will also be understood that all of the above-indicated preferred embodiments also apply to this method that uses at least one liquid and at least one solid.

Furthermore, it will also be understood that while the above preferred embodiments recite “incubating a mixture of at least two liquids” and “mixing said at least two liquids to obtain a mixture”, the invention is not limited to these method steps but may also be carried out as a method where the step of mixing has been omitted. For example, the invention also relates to a method for incubating a liquid or for incubating a mixture of at least two liquids, the method comprising passing said liquid or said mixture through a structure having multiple interconnected channels, thereby incubating said liquid or said mixture. It will also be understood that all of the above-indicated preferred embodiments also apply to this method. The invention also relates to a method for incubating a mixture of at least one liquid and at least one solid, the method comprising passing said mixture through a structure having multiple interconnected channels, thereby incubating said mixture. It will again be understood that all of the above-indicated preferred embodiments also apply to this method.

### BRIEF DESCRIPTION OF THE DRAWINGS

FIG. 1: A) Example of a UV profile of a breakthrough experiment. Elution volumes (EV) at the 5% and 50% signals are indicated by the dashed vertical lines. B) Different breakthrough profiles with corresponding EV/EV numbers and Bodenstein numbers. The beginning of the profile (which is crucial for virus inactivation) is reflected better in EV/EV number, than in Bodenstein number.

FIG. 2: Comparison between breakthrough experiments using acetone buffer and solvent/detergent-containing buffer. Listed are columns and superficial linear velocities at which pairs of experiments with different buffer systems were performed. Parameters of the breakthrough curves (i.e. EV_{5%}/EV_{50%} and Bodenstein numbers) were calculated for each buffer system. SD=combination of process fluid buffer and process fluid buffer with addition of solvent/detergent chemicals.

FIG. 3: Comparison between breakthrough experiments using acetone buffer and solvent/detergent-containing buffer. Each data point represents a pair of experiments with the same settings and different buffer systems: Water and 2% acetone (Acetone), process fluid buffer with addition of solvent/detergent chemicals (SD). Calculated parameters of the breakthrough curves EV_{5%/}EV_{50%} and Bodenstein number are very well correlated between buffer systems.

FIG. 4: Influence of column parameters and superficial linear velocity on Bodenstein number and EV_{1%}/EV_{50%}.

FIG. 5: Influence of column length on Bodenstein number and EV_{1%}/EV_{50%} as a measure of goodness for the RTD. The columns were packed with beads of the same batch. The column nomenclature used throughout the present figures follows the following principle: For example, for a column termed “JS_10_ceramic_HR_26/19.5_0.125_0.25 mm”, “10” is a unique integer number given to the column, “Ceramic” denotes the material of non-porous beads, “26” is the diameter of the column [mm], “19.5” is the height of the packed bed [cm], and “0.125_0.25 mm” is the particle size range indicated by the data from the bead manufacturer.

FIG. 6: Influence of superficial linear velocities on EV_{1%}/EV_{50%}.

FIG. 7: Partial least square (PLS) prediction model for RTD goodness parameters. Prediction is based on column length, column volume, superficial linear velocity, mean bead diameter and bead diameter range.

FIG. 8: PLS prediction model for RTD goodness parameters. Prediction is based on column length, column volume, mean bead diameter and bead diameter range.

FIG. 9: Influence of packing quality on RTD. Column JS_07 was hand-packed with many air bubbles (i.e., the packing quality was bad). At low superficial linear velocities, the badly packed column performed similarly as well packed columns with larger beads. However, at higher superficial linear velocities it underperformed.

FIG. 10: Lowering the limit of detection (LOD). Breakthrough experiments were performed using 10% acetone. The correlation between EV_{0.03%}/EV_{50%} (θ_{0.03}%) and EV_{1%}/EV_{50%} (θ_{1%}) was good, especially for well-packed columns.

FIG. 11: Comparison of columns packed with non-porous beads according to the present invention and known coiled flow inverters (CFI) in terms of Bodenstein numbers. Non-porous glass beads were used in the packed bed.

FIG. 12: Comparison of columns packed with non-porous beads according to the present invention and known coiled flow inverters (CFI) in terms of Bodenstein numbers. Non-porous ceramic beads were used in the packed bed.

FIG. 13: Comparison of columns packed with non-porous beads according to the present invention and known coiled flow inverters (CFI) in terms of Bodenstein numbers. Non-porous glass beads, PMMA plastic beads, or ceramic beads were used in the packed bed.

FIG. 14: Exemplary embodiment of the device for the preparation of a biopharmaceutical drug that can be used for virus inactivation.

FIG. 15: Pulse injection responses are smoothened derivatives of experimental breakthrough curves. The thick gray line represents the worst case elution profile when keeping the LOD point fixed in both dimensions. The thick black curve represents experimental data. The signal drop at the beginning is a consequence of flushing tubes on bypass before redirecting the sample through the column.

FIG. 16: A, B: Required residence time of the beginning of detectable breakthrough curve (LOD time) depending on limit of detection (LOD) and required viral reduction ratio assuming the same LRV is achieved in batch incubation mode in 60 min and a logarithmic virus reduction kinetics.

FIG. 17: Mixing of liquids prior to entering the continuous virus inactivation reactor (CVI). A: Mixing of two liquids. B: Mixing of three liquids. C: Mixing any number of liquids.

FIG. 18: Order of mixing of liquids prior to entering the virus inactivation reactor (CVI). A: Mixing of two liquids. B: Mixing of three liquids where two liquids are mixed before the third liquid is mixed with the resultant mixture. C: Mixing of any number of liquids prior to the admixture of additional liquids is possible.

FIG. 19: Exemplary process steps (and corresponding units of the reactor) upstream of virus inactivation (CVI). A: A surge tank is incorporated before virus inactivation. (left) Batch chromatography upstream of CVI. (middle) Counter-current loading chromatography upstream of CVI. (right) Simulated moving bed chromatography upstream of CVI. B: Seamless straight-through processing without a surge tank. (left) Batch chromatography. (middle) Counter-current loading chromatography. (right) Simulated moving bed chromatography.

FIG. 20: Exemplary process steps (and corresponding units of the reactor) downstream of virus inactivation. A: Solvent-detergent extraction in counter-current mode. B: Solvent-detergent extraction in co-current mode. C: Batch chromatography. D: Counter-current loading chromatography. E: Simulated moving bed chromatography.

FIG. 21: A: A large 1.75 L column has a much larger Bodenstein number (much narrower residence time distribution) than any (smaller) lab scale column, while some of the lab scale columns already completely surpass the coiled flow inverter reactors in terms of Bodenstein number. B: The same as panel A, except that the scales are in logarithmic form. C: The large 1.75 L column performs very well. In comparison, a smaller column (d=26 mm, I=19.5 cm) packed with the same batch of beads which achieved an EV_{1%}/EV_{50%} score in range of 0.88-0.92 and Bodenstein numbers in range of 800-1800.

FIG. 22: Picture of an illustrative example of a vibration device used for column packing. 1. Vibration motor, 2. Steel-frame, 3. Column, 4. Motion sensor, 5. Data recorder, 6. Power control

FIG. 23: Illustrative explanation of the superficial linear velocity [cm/h]: The superficial linear velocity is the linear velocity at which the fluid travels assuming that the structure (e.g. the packed bed of non-porous beads) is empty, e.g. not filled with beads. An exemplary structure (illustrated in the form of a cylinder that is filled with interconnected channels (B) or empty (A)) is shown in the Figure.

FIG. 24: Diagram of the CVI setup. The setup consists of two pumps, a mixer and the CVI.

FIG. 25: Concentration profile at the outlet for the CVI process. The plot shows the outlet concentration (C) normalized for the concentration at the inlet (C₀). The process is divided into two phases: a start-up (or latency) phase and a steady state phase. The start-up phase is represented by an initial 0%-concentration portion of the curve and a subsequent transition from 0 to 100% of the concentration. The start-up phase represents the displacement and washout of the liquid phase previously inside the CVIR until the concentration at the outlet matches the one at the inlet. The steady state phase is represented by the 100%-concentration portion of the curve. In this example the steady state starts before 2 V_{R}.

FIG. 26: Results of the virus titer for the CVI process at an incubation time of 30 and 60 min (in the left and right plot, respectively). The marker at 0 V_{R} represents the X-MuLV titer of the spiked test item before mixing with the S/D components. The markers at 1, 2, 3, 4 and 5 VR represent the X-MuLV titers at the outlet of the CVIR after operation for 1, 2, 3, 4 and 5 reactor volumes, respectively. The full markers show the virus titer and the open markers represent samples with titers below the LOD.

FIG. 27: The LRV for various samples collected during the continuous virus inactivation process with 30 and 60 min incubation time (top and bottom, respectively). The samples shown were taken after 1, 2, 3, 4, and 5 V_{R} of operation and also include a hold control (HC). The HC sample was drawn from the same syringe containing the spiked test time after the CVI was finished (after 5 V_{R}). The full-filled bars show the LRV data and the diagonal pattern-filled bars represent the minimum LRV due to samples falling below the LOD.

FIG. 28: The LRV for various samples collected during the traditional batch virus inactivation process. The samples shown were taken after 60 min of incubation and also include a hold control (HC). The HC sample was obtained by incubation of the spiked test item without S/D chemicals under the same conditions as the S/D-containing inactivation run. The full-filled bars show the LRV data and the diagonal pattern-filled bars represent the minimum LRV due to samples falling below the LOD.

FIG. 29: Results of the virus titer for the CVI process at an incubation time of 30 and 60 min (on the left and right plot, respectively). The marker at 0 VR represents the BVDV titer of the spiked test item before mixing with the S/D components. The markers at 1, 2, 3, 4 and 5 VR represent the BVDV titers at the outlet of the CVIR after operation for 1, 2, 3, 4 and 5 reactor volumes, respectively. The full markers show the virus titer and the open markers represent samples with titers that fell below the LOD.

FIG. 30: The LRV for various samples collected during the continuous virus inactivation process with 30 and 60 min incubation time (top and bottom, respectively). The samples shown were taken after 1, 2, 3, 4, and 5 V_{R} of operation and also include a hold control (HC). The HC sample was drawn from the same syringe containing the spiked test time after the CVI was finished (after 5 V_{R}). The full-filled bars show the LRV data and the diagonal pattern-filled bars represent the minimum LRV due to samples falling below the LOD.

FIG. 31: The LRV for various samples collected during the traditional batch virus inactivation process. The samples shown were taken after 60 min of incubation and also include a hold control (HC). The HC sample was obtained by incubation of the spiked test item without S/D chemicals under the same conditions as the S/D-containing inactivation run. The full-filled bars show the LRV data and the diagonal pattern-filled bars represent the minimum LRV due to samples falling below the LOD.

### DETAILED DESCRIPTION OF THE INVENTION

Definitions

Unless otherwise defined below, the terms used in the present invention shall be understood in accordance with their common meaning known to the person skilled in the art.

All publications, patents and patent applications cited herein are hereby incorporated by reference in their entirety for all purposes.

The term “residence time” as used herein generally refers to the amount of time that elapses from the moment a part of the liquid enters a part of processing equipment until the same part of the liquid exits the part of processing equipment. If the average linear velocity of a part of the liquid is high, the residence time is short. If the average linear velocity of a part of the liquid is low, the residence time is long. In one preferred embodiment of the invention, the term “residence time” refers to the amount of time that elapses from the moment a part of the liquid enters the structure having multiple interconnected channels until the same part of the liquid exits the structure having multiple interconnected channels. Alternatively, and more preferably, the term refers to the number of column volumes that pass from the moment a part of the liquid enters the structure having multiple interconnected channels until the same part of the liquid exits the structure having multiple interconnected channels. Residence time and elution volume are related by the following formula:

Elution volume (measured in column volumes)=residence time·column cross section·superficial linear velocity

When different parts of the liquid have different residence times, even though all parts of the liquid enter a part of processing equipment (e.g. the structure having multiple interconnected channels according to the invention) at the same time, the parts of the liquid are distributed with regard to their residence time. In other words, the different parts of the liquid show a distribution of residence times, which is also referred to as a “residence time distribution” or “RTD”. If there is a big difference between the flow velocities between the different parts of the liquid, the residence time distribution is broad; if there is a small difference in flow velocities between the different parts of the liquid, the residence time distribution is narrow. One of the advantages of the structure having multiple interconnected channels according to the invention is that it allows to obtain a narrow residence time distribution.

It is to be understood that the term “mixture of at least one liquid and at least one solid” defines that at the time when said at least one liquid and at least one solid were mixed, the solid was present in the solid state. This does not exclude the possibility that in said mixture of at least one liquid and at least one solid, the solid can dissolve, e.g. while further method steps according to the invention are carried out.

In accordance with all other embodiments of the invention, the mixture of two liquids or the mixture of at least one liquid and at least one solid can be an aqueous solution.

The term “interconnected channels” as used herein refers to channels in a structure that are accessible to fluids from the outside of said structure. At least some of the channels are interconnected with each other.

That way, when the structure is exposed to a liquid, the liquid can pass through the structure through those channels which are interconnected with each other. It is understood that the structure having multiple interconnected channels referred to in connection with the invention is such that it is suitable for passing the mixture of the at least two liquids in accordance with the invention through the structure.

The term “continuous” or “continuously” or “continuous-flow” as used herein in connection with the method or process of the invention or with steps thereof has the meaning that is commonly known in the art. It describes a method or process or steps thereof that occur(s) without interruption. If the term “continuous” or “continuously” or “continuous-flow” is used herein in accordance with particular method or process steps (e.g. with the step of mixing and passing according to the invention), it means that this step occurs without interruption. If the term “continuous” or “continuously” or “continuous-flow” is used herein in accordance with a method or process of the invention, it means that said method or process occurs without interruption. Preferably, where the method or process is carried out continuously, all method or process method steps are carried out continuously. Alternatively, it is also possible that only the output of the method or process is continuous, whereas parts of said method or process (e.g. particular method or process steps) are carried out discontinuously or semi-continuously. For example, a series of batch processes can deliver a continuous output over time, although the individual processes are operated discontinuously.

The term “non-porous beads” as used herein refers to any suitable non-porous beads that can be used for a packed bed of non-porous beads according to the invention. The “non-porous beads” can be spherical or irregularly shaped. In a preferred embodiment in accordance with all other embodiments of the invention, the non-porous beads are preferably spherical. The “non-porous beads” can, for instance, be made of any solid particulate material that is compatible with biopharmaceutical processing, e.g. plastics, glass or metal.

Non-porous beads are known in the art and are commercially available.

Glass beads are known in the art and can, for instance be made of silica glass. For example, glass beads can be purchased from Cospheric LLC.

Plastic beads are also known and can, for instance, be made of Poly(methyl methacrylate) (PMMA), polyethylene (PE), polypropylene (PP),or polystyrene (PS). For example, plastic beads can be purchased from Cospheric LLC, Altuglas Arkema, and Kisker Biotech.

Steel beads are also known in the art and can, for instance, be made of stainless steel. For example, steel beads can be purchased from Cospheric LLC.

The term “ceramic beads” as used herein refers to any ceramic beads that are suitable for forming a “packed bed of non-porous beads” according to the invention. For example, ceramic beads can be purchased from Kuhmichel Abrasiv GmbH.

The packed bed of non-porous beads according to the invention is not particularly limited and can, for instance, be contained in variously shaped containers, such as columns or reactors. The size of the container is not particularly limited, and can be selected based on the desired throughput and incubation time.

The term “inert” in connection with the non-porous beads of the invention has the meaning of the term that is known in the art. In a preferred embodiment, the inert non-porous beads are not functionalized in any way. Inert materials for the non-porous beads of the invention can be chosen by a person skilled in the art. For example, in a method or process of the invention, it will be possible to select appropriate known inert materials such that they do not or not substantially (e.g. not measurably) react with the liquid or mixture of liquids that is passed through the bed of beads. For example, the inert non-porous beads of the invention are preferably beads that not or not substantially chemically react with the liquid mixture of the present invention. The inert non-porous beads of the invention are preferably beads that do not add components to the liquid mixture. The inert non-porous beads of the invention preferably do not absorb or adsorb components from the liquid mixture.

The term “deviate from the mean particle diameter” by a given percentage as used herein refers to a deviation which depends on the mean particle diameter. For example, if beads with a mean particle diameter of 0.2 mm do not deviate from the mean particle diameter by more than 50%, 95% of the beads have a particle diameter of not more than 0.3 mm and not less than 0.1 mm. Similarly, if beads with a mean particle diameter of 0.2 mm do not deviate from the mean particle diameter by more than 20%, 95% of the beads have a particle diameter of not more than 0.24 mm and not less than 0.16 mm. For particles to be used in accordance with the present invention which are not spherical, the diameter refers to the longest axis of the particles.

The term “vibration treatment” as used herein refers to any treatment that involves vibration and which is suitable to increase the packing density of the packed bed of non-porous beads. For example, a vibrational device can be used for subjecting the packed bed of non-porous beads to vibration treatment.

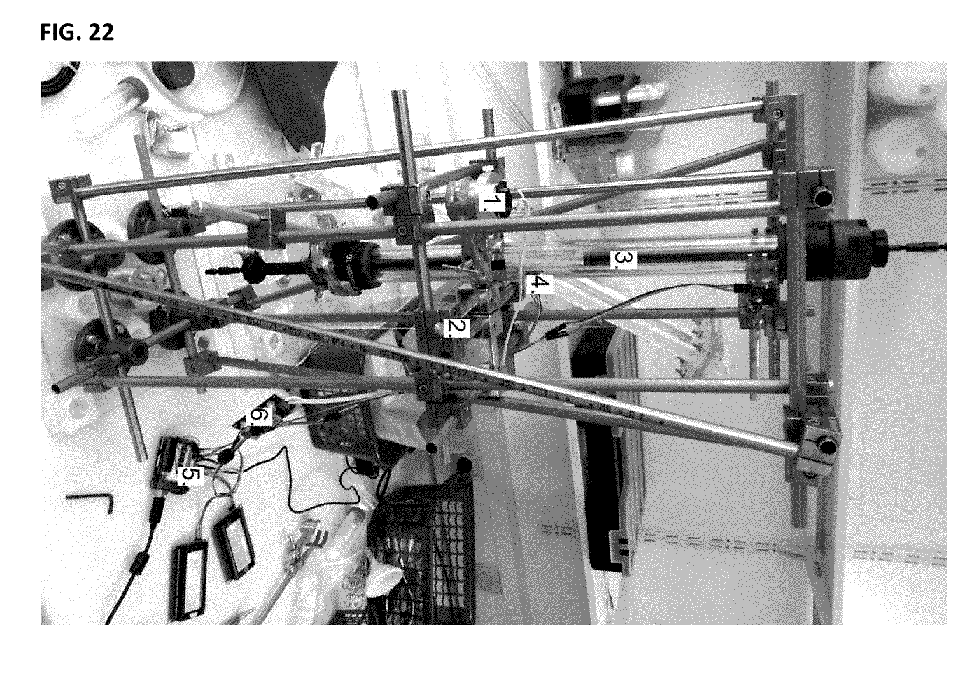

A preferable vibrational device contains a rack to which the column is immobilized. In an example of a vibration treatment using a vibrational device, the empty column is immobilized, and the beads are added during vibration. The rack is then vibrated using a vibration motor. Said motor can, for instance, be powered electrically or pneumatically. Packed beds of non-porous beads can be packed using a vibration frequency of less than 40 kHz, preferably of 1-10 kHz, an acceleration of less than 10 g, preferably 0-5 g, and a vibration amplitude of less than 5 mm, preferably up to 2 mm. An illustrative example of a vibrational device used for column packing is shown in FIG. 22.

The term “reactor” as used herein refers to any container or other structure that is suitable to contain fluids. The reactor can be used in order to allow the fluids to chemically react. However, in the present invention the term “reactor” also refers to reactors in which no chemical reaction occurs. It is understood that the reactor can be adjusted based on the intended use. For example, it is understood that a reactor that is used for virus inactivation will be suitable for virus inactivation. Likewise, if the reactor is used for the preparation of a biopharmaceutical drug it will be suitable for the preparation of that drug.

The term “3D-printed geometry” as used herein refers to any precast porous structure that is printed using a 3D printer.

The term “enveloped virus” as used herein has the meaning known to the person skilled in the art. For example, enveloped viruses can be Herpesviridae such as herpes simplex virus, varicella-zoster virus, cytomegalovirus or Epstein-Barr virus; Hepadnaviridae such as hepatitis B virus; Togaviridae such as rubella virus or alphavirus; Arenaviridae such as lymphocytic choriomeningitis virus; Flaviviridae such as dengue virus or bovine viral diarrhea virus (BVDV), hepatitis C virus or yellow fever virus; Orthomyxoviridae such as influenza virus A, influenza virus B, influenza virus C, isavirus or thogotovirus; Paramyxoviridae such as measles virus, mumps virus, respiratory syncytial virus, Rinderpest virus or canine distemper virus; Bunyaviridae such as California encephalitis virus or hantavirus; Rhabdoviridae such as rabies virus; Filoviridae such as Ebola virus or Marburg virus; Coronaviridae such as corona virus; Bornaviridae such as Borna disease virus; or Arteriviridae such as arterivirus or equine arteritis virus; Retroviridae such as Human Immunodeficiency Virus (HIV) or Xenotropic murine leukemia virus (X-MuLV), Human T-lymphotropic virus 1 (HTLV-1); Poxviridae such as Orthopoxvirus variolae (Variolavirus).

The term “solvent/detergent mixture” as used herein has the meaning known to the person skilled in the art. The term “solvent/detergent mixture” also relates to mixtures that contain only solvents or only detergents. In a preferred embodiment, the solvent/detergent mixture used in accordance with the invention contains at least one solvent other than water and at least one detergent. The number of different solvents and/or detergents contained in the mixture is not particularly limited. For example, the solvent/detergent mixture can be composed of tri-n-butyl phosphate, Polysorbate 80 and Triton X-100.

The term “solvent-detergent virus-inactivating treatment” as used herein has the meaning known to the person skilled in the art. In a preferred embodiment, solvent-detergent treatments can be used against enveloped viruses, e.g. by removing the lipid membrane of enveloped viruses. However, the “solvent-detergent virus-inactivating treatment” of the present invention is not limited thereto. For example, a “solvent-detergent virus-inactivating treatment” of the present invention can also include treatments of non-enveloped viruses, which, for instance, act by denaturing proteins on the surface of a virus such as a non-enveloped virus.

The term “Log10 reduction value” or “LRV” as used herein is a measure of the reduction of infectious virus particles in a fluid, defined as the logarithm (base 10) of the ratio of the infectious virus particle concentration before virus inactivation to the infectious virus particle concentration after virus inactivation. The LRV value is specific to a given type of virus. It is evident for a skilled person in the art that any Log10 reduction value (LRV) above zero is beneficial for improving the safety of methods and processes such as biopharmaceutical production methods and processes. In accordance with the invention, LRVs can be measured by any appropriate methods known in the art. Preferably, the LRVs referred to herein are LRVs as measured by plaque assay or as measured by the TCID₅₀ assay, more preferably as measured by the TCID₅₀ assay. These assays are known to the person skilled in the art. Preferably, the LRVs referred to in accordance with the invention are LRVs of an enveloped virus. For example, a “TCID₅₀ assay” as used herein refers to a tissue culture infectious dose assay. The TCID₅₀ assay is an end-point dilution test, wherein the TCID₅₀ value represents the viral concentration necessary to induce cell death or pathological changes in 50% of cell cultures inoculated.

The terms “flow rate” and “volumetric flow rate” as used in accordance with the invention are used interchangeably and refer to the volume of the mixture which passes through the structure having multiple interconnected channels according to the invention per amount of time. The volumetric flow rate (or flow rate) is preferably measured in mL/min. The volumetric flow rate (or flow rate) is constant regardless of the diameter of the tubing, regardless of the diameter of the structure having multiple interconnected channels (e.g. the column), and regardless of the pump piston. It is typically set by changing the pump speed to the desired flow rate. For example, if one or more pumps are used upstream of the structure having multiple interconnected channels, the volumetric flow rate (or flow rate) is the total volume displaced by said pumps per amount of time. For instance, piston pumps for example deliver a defined volume of fluid in each stroke of the piston. Syringe pumps are driven by a linear motor. Using the syringe diameter and the distance the syringe piston is pushed by the motor, the displaced volume per amount of time can be calculated. Alternatively, the flow rate can also be measured by flow meters which are known in the art.

Generally, a “linear velocity” is defined as a flow rate divided by the cross-sectional area of the structure the liquid is passing through:

linear velocity=(volumetric flow rate)/(cross-sectional area)

The term “linear velocity” as used in connection with the structures of the invention refers to the volumetric flow rate, divided by the cross-sectional area of the structure having multiple interconnected channels. The cross section may typically be circular, i.e. the cross section is a circle.

In a structure having multiple interconnected channels of the invention such as a packed bed of non-porous beads, two different linear fluid velocities can be distinguished:

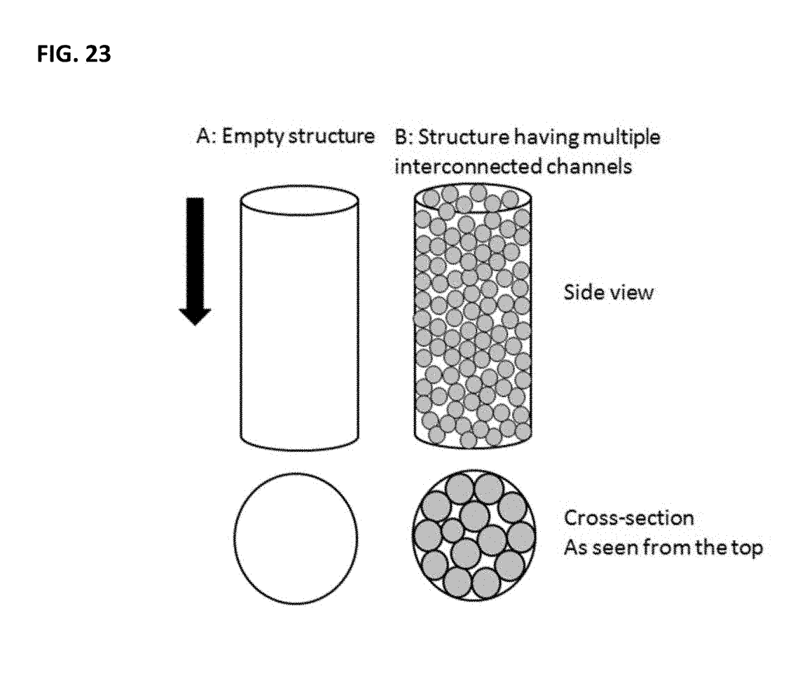

a) Superficial linear velocity (preferably indicated in [cm/h]): The superficial linear velocity is the linear velocity at which the fluid travels assuming that the structure (e.g. the packed bed of non-porous beads) is empty, e.g. not filled with beads. An exemplary structure (illustrated in the form of a cylinder that is filled with interconnected channels (B) or empty (A)) is shown in FIG. 23.

b) Interstitial linear velocity (preferably indicated in [cm/h]): The interstitial velocity is the actual fluid velocity through the structure having multiple interconnected channels (e.g. through the packed bed of non-porous beads). Since the fluid can only flow through the interconnected channels (e.g. around the beads), the interstitial velocity is always higher than the superficial velocity.

Unless stated otherwise, all occurrences of the term “linear velocity” as used herein refer to the superficial linear velocity. The superficial linear velocity can be calculated by dividing the flow rate (or volumetric flow rate) by the cross-sectional area of the structure having multiple interconnected channels, assuming that the structure is empty.

The term “limit of detection” or “LOD” as used herein refers to the lowest detectable share of a substance, e.g. to the lowest detectable share of beads in suspension. The term “limit of detection time” or “LOD time” as used herein refers to the time point at which the signal emanating from a substance, e.g. from a tracer substance such as beads in suspension, surpasses the limit of detection (LOD).

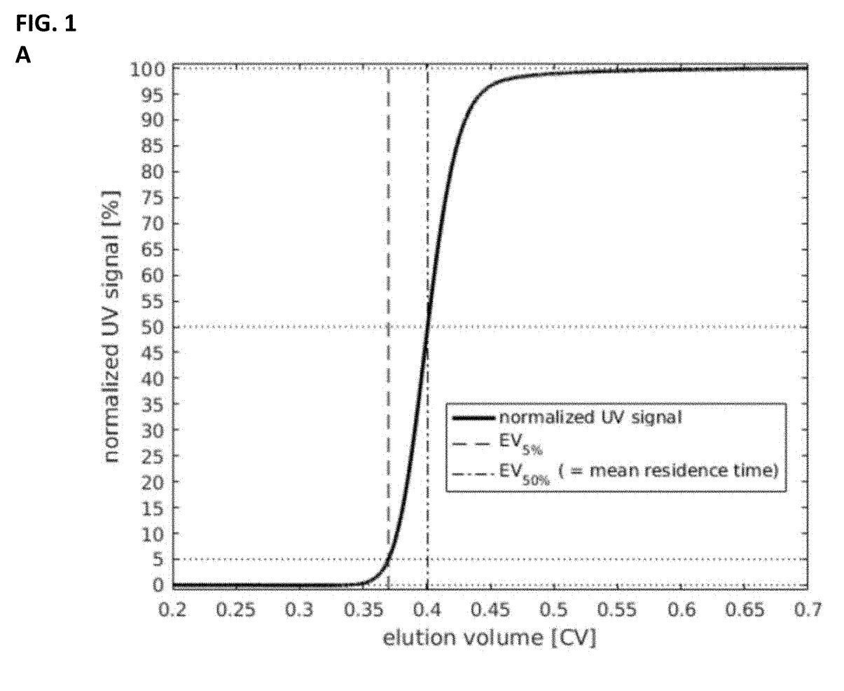

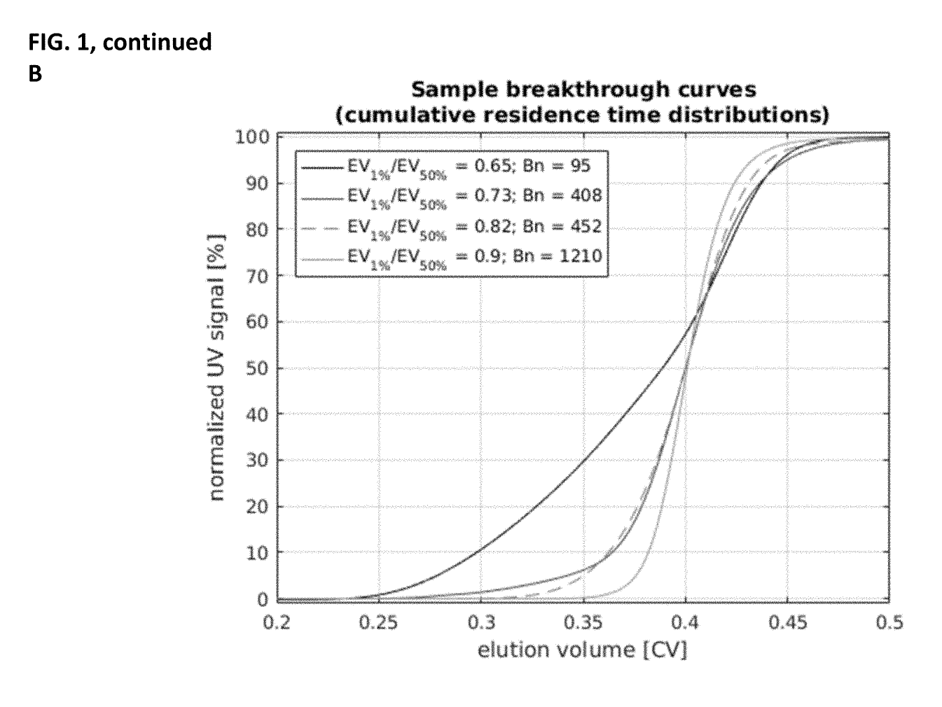

The term “Bodenstein number” as used herein has the meaning known to the person skilled in the art. It is, for example, described in Levenspiel, Chemical Reaction Engineering, 3rd ed., John Wiley & Sons, 1999 (Ref. 8), which is incorporated by reference in its entirety for all purposes. The Bodenstein number is dimensionless and characterizes the backmixing within a system. Thus, the Bodenstein number can indicate the extent to which liquid volumes or compounds backmix. For example, a small Bodenstein number indicates a large degree of backmixing, whereas a large Bodenstein number indicates a small degree of backmixing. As will be known to a person skilled in the art, the Bodenstein number can be used as a measure of the residence time distribution and can be determined by methods known in the art. In the present invention, the Bodenstein number can preferably be calculated by fitting the function F to breakthrough curves (as exemplified in the examples; see e.g. FIG. 1A), where F(EV) represents the integral of a Gaussian peak (e.g. a UV signal of a tracer substance added to the mixture that is passed through the structure having multiple interconnected channels according to the invention) and Bo represents the Bodenstein number, EV represents the elution volume at a given time point and EV_{50%} represents the elution volume at the mean residence time:

$$F ( EV ) = \frac{1}{2} ( erf ( \frac{1}{2} \sqrt{Bo} ( \frac{EV}{{EV}_{50 \%}} - 1 ) ) + 1 )$$

FIG. 1B represents a few different breakthrough profiles with corresponding EV/EV numbers and Bodenstein numbers. The Figure demonstrates that the beginning of the profile (which is crucial in particular for virus inactivation) is reflected much better in EV/EV number, than in Bodenstein number.

In accordance with the present invention, each occurrence of the term “comprising” may optionally be substituted with the term “consisting of”.

In the following we will describe specific embodiments of the invention, but the invention is not limited thereto. Moreover, any of the below embodiments can be combined with any of the other below embodiments according to the present invention.

The structure having multiple interconnected channels to be used in accordance with the invention can be a monolith or a precast structure such as a 3D printed geometry, but it is preferably a packed bed of non-porous beads. Thus, particularly the packed bed of non-porous beads can be combined with any other embodiments of the present invention.

The packed bed of non-porous beads to be used in accordance with the present invention can be contained in variously shaped containers, such as columns and/or reactors. Preferably, the packed bed of non-porous beads completely fills the container, i.e. it does not leave any large gaps. Preferably, the container comprises at least an inlet and at least an outlet that are positioned at opposite ends of the container. That way, a fluid can enter the container through the inlet, pass through the packed bed of non-porous beads, and exit the container through the outlet. Preferably, the container is a column.

The container for the packed bed of non-porous beads to be used in accordance with the present invention can have any shape, e.g. it can have a circular base, an angular base, or a rectangular base. Preferably, the container has a circular base. In a particularly preferred embodiment of the invention, the packed bed of non-porous beads to be used in accordance with the present invention is contained in a column with a circular base.

The length of the packed bed of non-porous beads to be used in accordance with the present invention is not particularly limited, and can be adjusted taking into account the desired throughput, the desired superficial linear velocity and the desired mean residence time of the liquid. In particular, the length of the packed bed of non-porous beads can be selected based on the desired superficial linear velocity and the desired mean residence time of the liquid. For example, if the desired superficial linear velocity is 20 cm/h and the porosity of the packed bed of non porous beads is assumed to equal 0.4, and the desired mean residence time is at least 1 h, then the length of the packed bed of non-porous beads needs to be at least 50 cm. If the desired superficial linear velocity is 20 cm/h, and the desired mean residence time is at least 3 h, then the length of the packed bed of non-porous beads needs to be at least 150 cm. In a preferred embodiment, the desired superficial linear velocity is about 20 cm/h and the desired mean residence time is at least 1 hour, so that the packed bed of non-porous beads needs to have a length of at least 50 cm. The inventors have found that the longer the packed bed of non-porous beads to be used in accordance with the present invention, the narrower the residence time distribution of a liquid that is passed through the bed of non-porous beads. Thus, if the packed bed of non-porous beads to be used in accordance with the present invention is longer (e.g. if it has a length of at least 5 cm, or at least 10 cm, or at least 20 cm, or at least 30 cm, or at least 50 cm, or at least 70 cm, or at least 100 cm) this is advantageous for a narrow residence time distribution.

The width or diameter of the packed bed of non-porous beads to be used in accordance with the present invention is not particularly limited, and it can be selected based on the desired throughput, the desired superficial linear velocity and the desired mean residence time of the liquid. It is apparent to the skilled person that the width or diameter of the packed bed of non-porous beads will be selected by taking the size of the beads into account. In other words, the width or diameter of the packed bed of non-porous beads will be chosen such that it is sufficient in order to accommodate the beads. In a preferred embodiment of the invention, the column diameter is 5 mm, preferably at least 10 mm.

The volume of the packed bed of non-porous beads to be used in accordance with the present invention is not particularly limited, and it can be selected taking into account the desired throughput, the desired superficial linear velocity and the desired mean residence time of the liquid. However, the inventors have surprisingly found that large volumes of the packed bed of non-porous beads provide for narrower residence time distributions than small volumes when a liquid is passed through the bed of non-porous beads. Thus, large volumes of the packed bed of non-porous beads are preferred, e.g. void volumes of at least 10 mL, preferably at least 40 mL, more preferably at least 150 mL, still more preferably at least 470 mL and still more preferably at least 700 mL.

The non-porous beads forming the packed bed of non-porous beads for use in accordance with the present invention can have various mean particle diameters. It will be understood that the diameter of the non-porous beads can easily be selected such that the interconnected channels formed by the spaces between the beads are suitable for the components (e.g. biopharmaceutical drugs) of the liquid (e.g. of the mixture used according to the present invention) to pass through the packed bed of non-porous beads. On the other hand, the inventors have surprisingly found that the smaller mean particle diameter of the beads forming the packed bed of non-porous beads according to the present invention, the narrower the residence time distribution of a liquid that is passed through the packed bed. Thus, the beads to be used in accordance with the present invention are preferably in the range of 0.05 mm to 1 mm, more preferably in the range of 0.05 mm to 0.6 mm, still more preferably in the range of 0.05 mm to 0.5 mm and most preferably in the range of 0.05 mm to 0.3 mm. Moreover, the inventors have surprisingly found that the more homogenous the mean particle diameter of the beads to be used in accordance with the present invention, the narrower the residence time distribution of a liquid that is passed through the packed bed of non-porous beads. Thus, the beads to be used in accordance with the present invention preferably do not deviate from the mean particle diameter by more than 50%, more preferably not more than 35%, most preferably not more than 20%.

Preferably, the non-porous beads forming the packed bed of non-porous beads for use in accordance with the present invention are inert.

Preferably, the non-porous beads forming the packed bed of non-porous beads for use in accordance with the present invention are spherical.

The non-porous beads can be packed by various means to form the packed bed of non-porous beads for use in accordance with the present invention. The inventors have found that differences in packing quality affect the flow paths of the liquids that are passed through the packed bed of non-porous beads, and thus the residence time distribution.

Exemplary means to pack the non-porous beads for use according to the present invention are dry packing or wet packing, with and without vibration treatment. The liquid packing can be by gravity or under flow. A preferred means to pack the non-porous beads for use according to the present invention is packing vibration treatment. Also preferred is wet packing, more preferably in combination with vibration treatment. Packing quality can be determined e.g. by determining the residence time distribution of a liquid that is passed through the packed bed of non-porous beads. A narrow residence time distribution is indicative of good packing quality, a broad residence time distribution is indicative of bad packing quality.

The method for incubating a mixture of at least two liquids in accordance with the present invention comprises the mixing of said at least two mixtures to obtain a mixture and the passing of said mixture through a structure having multiple interconnected channels, thereby incubating said mixture. Preferably, said mixing and passing is carried out continuously. Surprisingly, the inventors have found that when passing a liquid such as a mixture of at least two liquids according to the invention through the structure having multiple interconnected channels in order to incubate said liquid (e.g. said mixture), the incubation takes place with a particularly narrow residence time distribution. Such narrow residence time distribution is advantageous for all types of continuously operating processes wherein liquids have to be mixed and incubated for defined periods of time, because it allows to choose the incubation times more precisely.

In the method for incubating in accordance with the invention, the superficial linear velocity of the mixture that is passed through a structure having multiple interconnected pores is not particularly limited, and it can be selected based on the desired throughput. The inventors have found that lower superficial linear velocities of a liquid of the invention (e.g. the mixture used in accordance with the invention) that is passed through a structure having multiple interconnected channels provide for narrower residence time distributions than higher superficial linear velocities. Thus, the superficial linear velocity in the method for incubating in accordance with the present invention is preferably equal to or lower than 600 cm/h, or equal to or lower than 300 cm/h, or equal to or lower than 200 cm/h, or equal to or lower than 100 cm/h, or equal to or lower than 50 cm/h, or equal to or lower than 20 cm/h. Most preferably, the superficial linear velocity is equal to or lower than 50 cm/h.

As will be known to a person skilled in the art, the Bodenstein number can be used as a measure of the residence time distribution. A small Bodenstein number is indicative of a broad residence time distribution, and a large Bodenstein number is indicative of a narrow residence time distribution. As described above, in the method for incubating according to the present invention, it is very preferable that the mixture passing through a structure having multiple interconnected channels has a narrow residence time distribution. Accordingly, in the method for incubating according to the present invention, it is preferable that the Bodenstein number of the mixture passing through a structure having multiple interconnected channels is equal to or higher than 50, more preferably equal to or higher than 300, still more preferably equal to or higher than 400, still more preferably equal to or higher than 500, still more preferably equal to or higher than 600, most preferably equal to or higher than 800.

One example for a process wherein liquids (e.g. mixtures of at least two liquids) are incubated for a defined period of time while being passed through a structure having multiple interconnected channels is continuous virus inactivation. Thus, in a preferred embodiment of the present invention, the method for incubating according to the present invention is for continuous virus inactivation. In this preferred embodiment, a first liquid of said at least two liquids is a liquid potentially containing a virus, and a second liquid of said at least two liquids comprises a virus-inactivating agent. When incubating the mixture of a liquid potentially containing a virus and a liquid comprising a virus-inactivating agent, incubation time can be selected such that it is long enough to achieve sufficient Log10 Reduction Value (LRV) for a given virus. On the other hand, incubation time is preferably also selected such that it is short enough to ensure that other components that may be contained in the liquids (e.g. a biopharmaceutical) are not damaged by the virus-inactivating agent. If for all (or at least a majority of) parts of the liquid (e.g. a mixture of at least two liquids) the incubation time is similar, then a suitable incubation time that is neither to short, nor too long can be achieved more easily. Thus, the narrow residence time distributions which are obtained according to the invention are advantageous in that they, for instance, allow to select such suitable incubation times.

In biopharmaceutical production processes, viruses in the mixture containing the biopharmaceutical drug are typically inactivated to ensure that after formulation of the biopharmaceutical drug into a pharmaceutical composition, the pharmaceutical composition does not pose any harm to patients. Thus, the method or process for virus inactivation according to the present invention is particularly useful in biopharmaceutical production processes. Accordingly, in a preferred embodiment of the method or process for virus inactivation according to the present invention, the first liquid of the mixture of at least two liquids that is passed through a structure having multiple interconnected channels comprises a biopharmaceutical drug. Accordingly, the present invention also relates to a method for preparing a biopharmaceutical drug, wherein said biopharmaceutical drug is recovered after performing the method for incubating according to the present invention.

Methods for recovering a biopharmaceutical drug which can suitably be used after performing the method for incubating according to the present invention are well known to a person skilled in the art. For example, various chromatography methods can be used to recover a biopharmaceutical drug. Such methods can be selected by a person skilled in the art taking into account the properties of the biopharmaceutical drug, the source from which it is obtained (e.g. recombinantly or from other sources such as from human plasma) and the desired biopharmaceutical application (e.g. whether it will be administered subcutaneously or intravenously, etc.).

Biopharmaceutical drugs in accordance with the invention are not particularly limited. They include both recombinant biopharmaceutical drugs and biopharmaceutical drugs from other sources such as biopharmaceutical drugs obtained from human plasma. Biopharmaceutical drugs in accordance with the invention include, without limitation, blood factors, immunoglobulins, replacement enzymes, vaccines, gene therapy vectors, growth factors and their receptors. Preferred blood factors include factor I (fibrinogen), factor II (prothrombin), Tissue factor, factor V, factor VII and factor Vila, factor VIII, factor IX, factor X, factor XI, factor XII, factor XIII, von Willebrand Factor (VWF), prekallikrein, high-molecular-weight kininogen (HMWK), fibronectin, antithrombin III, heparin cofactor II, protein C, protein S, protein Z, plasminogen, alpha 2-antiplasmin, tissue plasminogen activator (tPA), urokinase, plasminogen activator inhibitor- 1 (PAI1), and plasminogen activator inhibitor-2 (PAI2). The blood factors that can be used in accordance with the present invention are meant to include functional polypeptide variants and polynucleotides that encode the blood factors or encode such functional variant polypeptides. Preferred immunoglobulins include immunoglobulins from human plasma, monoclonal antibodies and recombinant antibodies. The biopharmaceutical drugs in accordance with the invention are preferably the respective human or recombinant human proteins or functional variants thereof.

After recovering the biopharmaceutical drug obtained by the method for preparing a biopharmaceutical drug according to the present invention, the biopharmaceutical drug can be formulated into a pharmaceutical composition. Such pharmaceutical composition can be prepared in accordance with known standards for the preparation of pharmaceutical compositions. For example, the composition can be prepared in a way that it can be stored and administered appropriately, e.g. by using pharmaceutically acceptable components such as carriers, excipients or stabilizers. Such pharmaceutically acceptable components are not toxic in the amounts used when administering the pharmaceutical composition to a patient.

In connection with all embodiments of the method or process for virus inactivation according to the present invention, said method or process is preferably a method or process for continuous virus inactivation.

Particularly in the method or process for virus inactivation according to the present invention, it can be advantageous to monitor the residence time of the liquid in the structure having multiple interconnected channels, and its residence time distribution. Such monitoring would allow recognizing if any given part of the liquid of the mixture that is passed through the structure having multiple interconnected channels does not spend sufficient time in the structure having multiple interconnected channels. In the method for continuous virus inactivation according to the present invention, it can be advantageous to recognize if any given part of the liquid of the mixture that is passed through the structure having multiple interconnected channels does not spend sufficient time in the structure having multiple interconnected channels, because in such a case the first liquid (e.g. comprising a biopharmaceutical drug) may not be exposed to the virus-inactivating agent for long enough to achieve the desired Log10 reduction value for a given virus. In such a case, the skilled person could modify the method or process for virus inactivation in accordance with the invention, e.g. by increasing the length of the structure having multiple interconnected channels and/or by reducing the superficial linear velocity.

In the method or process for virus inactivation according to the present invention, in order to monitor the residence time of the liquid in the structure having multiple interconnected channels and its residence time distribution, a tracer sample can be periodically spiked-in upstream of the structure having multiple interconnected channels. For example, a tracer sample can be periodically spiked into a first liquid, which is subsequently mixed with a second liquid and optionally further liquids. Alternatively, a tracer sample can be periodically spiked into the mixture of at least two liquids and mixed with said mixture. Subsequently, when the mixture comprising the tracer is passed through the structure having multiple interconnected channels according to the present invention, the concentration of the tracer in the mixture can be monitored upstream and downstream of the structure having multiple interconnected channels. This monitoring can be carried out by any suitable method. Suitable analytic methods are known to a person skilled in the art. Such methods can be based on e.g. fluorescence detection, absorbance detection or nuclear magnetic resonance (NMR). Accordingly, in a preferred embodiment, the method or process for virus inactivation according to the present invention comprises a step of monitoring the residence time and residence time distribution of the liquid (e.g. the mixture of at least two liquids used according to the invention) in the structure having multiple interconnected channels, said step comprising the periodical spiking of a tracer sample into said liquid (e.g. into said mixture of at least two liquids used according to the invention) and the monitoring of the concentration of said tracer in the said liquid (e.g. in said mixture of at least two liquids used according to the invention) upstream and downstream of the structure having multiple interconnected channels. This step is advantageous in that it allows to monitor the quality of the structure having multiple interconnected channels during a continuous production process, e.g. in order to detect potential clogging or other disturbances of the structure. Further, this step is also advantageous in that it allows to monitor whether the residence time distribution of the structure having multiple interconnected channels remains sufficiently narrow in order to provide the desired LRV, e.g. an LRV of 4.

In the method or process for virus inactivation according to the present invention, it is preferred that the virus-inactivating agent is a solvent/detergent mixture suitable for solvent/detergent virus-inactivating treatment. The solvent/detergent mixture according to the invention is not particularly limited. For example, the solvent/detergent mixture can comprise a single organic solvent and a plurality of surfactants, a plurality of organic solvents and a single surfactant, or a plurality of organic solvents and a plurality of surfactants. It is understood that the type of detergent and/or solvent and their respective concentrations can appropriately be chosen by a skilled person, by taking into account, for instance, the potential viruses present in the liquid, the desired LRV, the properties of the biopharmaceutical drug and the characteristics of the manufacturing process of the biopharmaceutical drug (e.g. at which temperature the inactivation will be carried out). Typically, the final concentrations of an organic solvent and a single surfactant during the incubation in accordance with the invention is about 0.1% (v/v) to about 5% (v/v) of organic solvent and about 0.1% (v/v) to about 10% (v/v) of surfactant. When a plurality of surfactants are used, the final concentration of an organic solvent is about 0.1% (v/v) to about 5% (v/v), the final concentration of one surfactant is about 0.1% (v/v) to about 10% (v/v), about 0.5% (v/v) to about 5% (v/v), or about 0.5% (v/v) to about 1.0% (v/v), and the final concentration of the remainder of surfactants is about 0.1% (v/v) to about 5% (v/v), about 0.1% (v/v) to about 1.0% (v/v), or about 0.2% (v/v) to about 4% (v/v).

In one embodiment of the present invention, the solvent/detergent mixture comprises tri(n-butyl) phosphate and polyoxyethylene octyl phenyl ether (also known as, e.g. TRITON® X-100). In another embodiment, the solvent/detergent mixture comprises tri(n-butyl) phosphate and polyoxyethylene (80) sorbitan monooleate (also known as, e.g. Polysorbate 80 or TWEEN® 80).

In another embodiment of the present invention, the solvent/detergent mixture comprises tri(n-butyl) phosphate, polyoxyethylene octyl phenyl ether (TRITON® X-100), and polyoxyethylene (80) sorbitan monooleate (also known as, e.g. polysorbate 80 or TWEEN® 80).

In a preferred embodiment of the method or process for virus inactivation according to the present invention, a first liquid comprising a biopharmaceutical drug and a second liquid comprising a solvent/detergent mixture suitable for solvent/detergent virus-inactivating treatment are mixed and the mixture is subsequently passed through a structure having multiple interconnected channels. As will be apparent to a person skilled in the art, the concentrations of one or more components of the mixture for solvent/detergent virus-inactivating treatment can be monitored in the mixture that is passed through the structure having multiple interconnected channels, e.g. upstream of the structure having multiple interconnected pores, or downstream of the structure having multiple interconnected pores. For example, one or more components of the mixture that is passed through the structure with multiple interconnected channels can be tracked using UV VIS spectroscopy and Fourier transform infra-red (FTIR) spectroscopy, which are well known to a person skilled in the art.

Alternatively, in the method for continuous virus inactivation according to the present invention, the virus-inactivating agent can be an acidic solution suitable for low pH virus-inactivating treatment. An acidic solution suitable for low pH virus-inactivating treatment can comprise any inorganic or organic acid suitable for low pH virus-inactivating treatment.

In the method or process for virus inactivation according to the present invention, it is preferable that the method achieves at least a 1 Log10 reduction value (LRV) for at least one virus, or at least a 2 Log10 reduction value (LRV) for at least one virus, or at least a 3 Log10 reduction value (LRV) for at least one virus, or at least a 4 Log10 reduction value (LRV) for at least one virus, or at least a 5 Log10 reduction value (LRV) for at least one virus, or at least a 6 Log10 reduction value (LRV) for at least one virus, or at least a 7 Log10 reduction value (LRV) for at least one virus, or at least a 8 Log10 reduction value (LRV) for at least one virus, most preferably at least a 4 Log10 reduction value (LRV) for at least one virus. Of course, it is evident for a skilled person in the art that any Log10 reduction value (LRV) for at least one virus is beneficial, because it improves the safety of e.g. the biopharmaceutical production process. The LRVs referred to in accordance with the invention are preferably LRVs of an enveloped virus.

The Log10 reduction value (LRV) that is achieved by the method for continuous virus inactivation according to the present invention is determined as known to a person skilled in the art. For example, the LRV can be determined by determining the infectious virus particle concentration in a liquid before and after subjecting the liquid to the method for continuous virus inactivation according to the present invention. More specifically, the LRV can be determined by determining the infectious virus particle concentration in a first liquid, mixing the first liquid with a second liquid comprising a virus-inactivating agent in order subject the first liquid to the method for continuous virus inactivation according to the present invention, and determining the infectious virus particle concentration in the mixture of the first liquid and the second liquid after performing the method for continuous virus inactivation according to the present invention. Following determination of the infectious virus particle concentrations before and after virus inactivation, the LRV for any given virus can be determined by calculating the logarithm (base 10) of the ratio of the infectious virus particles before virus inactivation (=infectious virus particle concentration before virus inactivation*volume before virus inactivation, e.g. volume of first liquid) to the infectious virus particles after virus inactivation (=infectious virus particle concentration after virus inactivation, e.g. in mixture of first and second liquid*(volume after virus inactivation, e.g. volume of first liquid+volume of second liquid)).

The skilled person is aware of numerous methods for determining the infectious virus particle concentrations in a liquid. For example, and without limitation, infectious virus particle concentrations in a liquid can preferably be measures by plaque assay or by the TCID₅₀ assay, more preferably by the TCID₅₀ assay..

As will be known to a person skilled in the art, virus inactivation by mixing a liquid with a solvent/detergent mixture suitable for solvent/detergent virus-inactivating treatment and virus inactivation by mixing a liquid with an acidic solution suitable for low pH virus-inactivating treatment are particularly effective for inactivating enveloped viruses. Thus, in a preferred embodiment, the method or process for virus inactivation according to the present invention is for continuous virus inactivation of enveloped viruses.

The present invention also discloses a device for the preparation of a biopharmaceutical drug in accordance with the methods of the present invention. Said device comprises a packed bed of non-porous beads. Since the device including the packed bed of non-porous beads is preferably used in a method according to the present invention, the packed bed of beads comprised in the device preferably has the same embodiments as the packed bed of non-porous beads for use according to the present invention as described above.

In the method for preparing a biopharmaceutical drug according to the present invention, a first liquid comprising a biopharmaceutical drug and a second liquid comprising a virus-inactivating agent are mixed and the mixture is subsequently passed through a structure having multiple interconnected channels. In an optional embodiment of the present invention, a static mixer can be used for mixing the at least two liquids before passing the mixture through the structure having multiple interconnected channels. Thus, in one embodiment of the invention the device for the preparation of a biopharmaceutical drug according to the present invention comprises a static mixer. In a preferred aspect of this embodiment, said static mixer is located upstream of the packed bed of non-porous beads. In a further preferred aspect of this embodiment, said static mixture is a T-junction mixer.

In the method for preparing a biopharmaceutical drug according to the present invention, the mixture of at least two liquids may contain debris, e.g. cellular debris, or other insoluble components from the upstream biopharmaceutical production process. Thus, it may be desired to remove said insoluble components from the mixture e.g. by filtration. Accordingly, in one embodiment of the invention the device for the preparation of a biopharmaceutical drug according to the present invention comprises a filter. In a preferred aspect of this embodiment, said filter is located upstream of the packed bed of non-porous beads. In an even more preferred aspect of this embodiment, the filter is located upstream of the packed bed of non-porous beads, and downstream of a mixer, such as a T-junction mixer or a dynamic mixer. The pore size of the filter is not particularly limited and will be selected by a person skilled in the art, e.g. by taking into account the size of the biopharmaceutical drug that needs to pass the filter and the size of the components (e.g. the cellular debris or other insoluble components from the upstream biopharmaceutical production process) that should be removed from the process. In a preferred embodiment, the filter has a pore size of 0.2 μm.

In another embodiment in accordance with the above embodiments, the device for the preparation of a biopharmaceutical drug according to the present invention is a continuous-flow reactor, which comprises a packed bed of non-porous beads. As will be apparent to a person skilled in the art, the reactor in accordance with the present invention can be combined with all other embodiments of the device for the preparation of a biopharmaceutical drug according to the present invention. For example, the reactor can comprise a mixer such as a T-junction mixer upstream of the packed bed of non-porous beads. Alternatively, the reactor can comprise a filter, e.g. a filter with a pore size of 0.2 μm, upstream of the packed bed of non-porous beads. As another alternative, the reactor can comprise a filter, e.g. a filter with a pore size of 0.2 μm, upstream of the packed bed of non-porous beads, and a mixer such as a T-junction mixer upstream of the filter. In a preferred aspect of this embodiment, the reactor is a column, which comprises a filter, e.g. a filter with a pore size of 0.2 μm, upstream of the packed bed of non-porous beads, and a static mixer such as a T-junction mixer upstream of the filter.

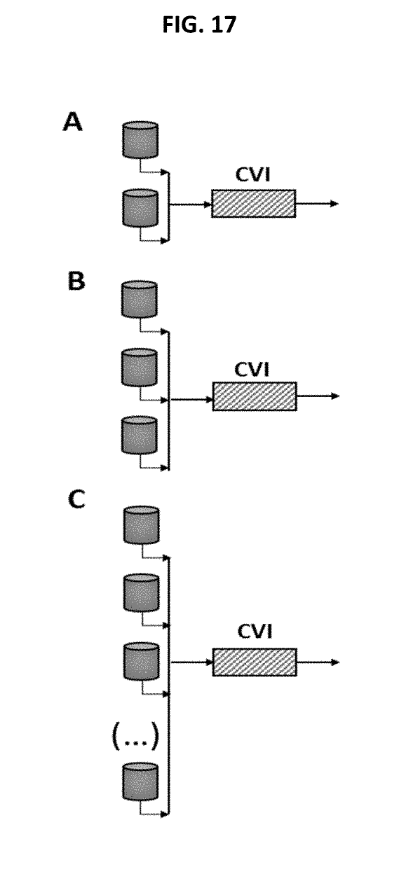

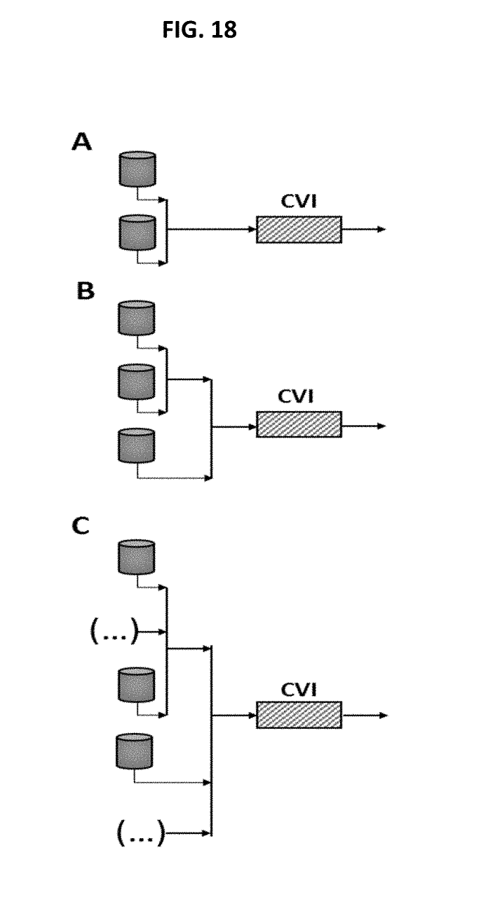

In an embodiment in accordance with all other embodiments of the invention, the continuous-flow reactor is suitable for continuous virus inactivation. The continuous-flow reactor for continuous virus inactivation of the invention preferably comprises mixers for two liquids, of three liquids, or of four or more liquids which are connected to the packed bed of non-porous beads. These mixers are positioned upstream of the packed bed of non-porous beads such that the liquids can be mixed prior to entering the packed bed of non-porous beads. Non-limiting examples of such mixing configurations are given in FIG. 17. The order of mixing is not particularly limited. For example, three liquids can be mixed in a way that two liquids are mixed before the third liquid is mixed with the resultant mixture, or any number of liquids can be mixed prior to the admixture of additional liquids. Non-limiting examples of such mixing configurations are given in FIG. 18.

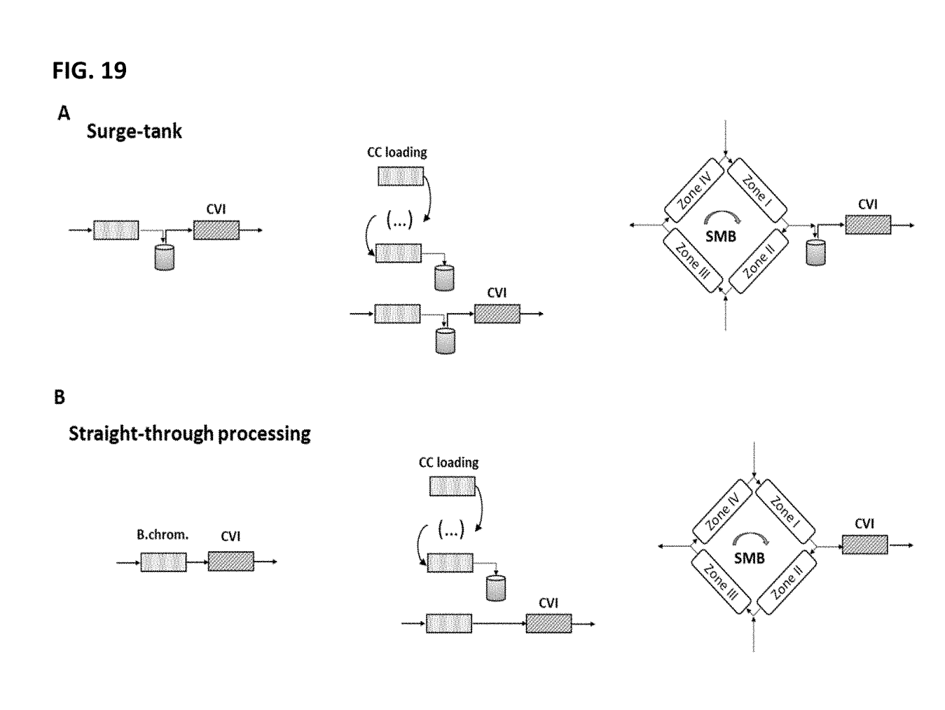

The continuous-flow reactor for continuous virus inactivation of the invention preferably comprises further units upstream of the packed bed of non-porous beads, which can include a surge tank. In non-limiting embodiments, the surge tank can be connected to a batch chromatography unit upstream of the surge tank, or to a unit for counter-current loading chromatography upstream of the surge tank, or to a unit for simulated moving bed chromatography upstream of the surge tank. Non-limiting examples of such units upstream of the packed bed of non-porous beads are shown in FIG. 19 A. Alternatively, the continuous-flow reactor for continuous virus inactivation of the invention preferably comprises further units upstream of the packed bed of non-porous beads, which include a unit for seamless straight-through processing without a surge tank. In non-limiting embodiments, the unit for seamless straight-through processing can be a batch chromatography unit, a unit for counter-current loading chromatography, or a unit for simulated moving bed chromatography. Non-limiting examples of such units upstream of the packed bed of non-porous beads are shown in FIG. 19 B.

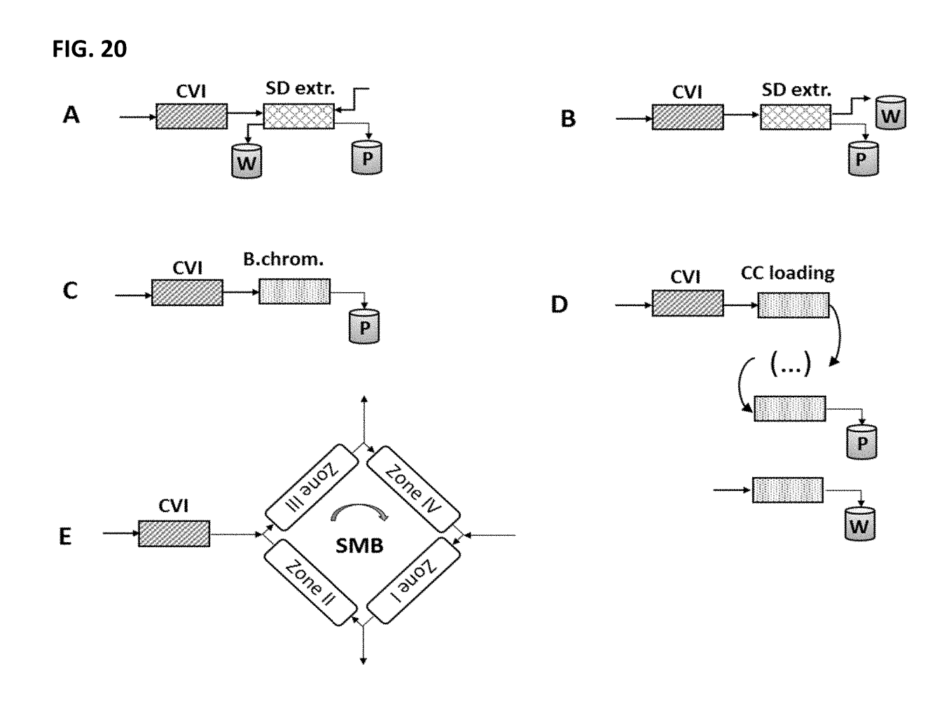

The continuous-flow reactor for continuous virus inactivation of the invention preferably comprises further units downstream of the packed bed of non-porous beads, including but not limited to a unit for solvent-detergent extraction in counter-current mode, a unit for solvent-detergent extraction in co-current mode, a batch chromatography unit, a unit for counter-current loading chromatography and a unit for simulated moving bed chromatography. Non-limiting examples of such units downstream of the packed bed of non-porous beads are shown in FIG. 20.

It will be understood that the above-described units of the reactor for continuous virus inactivation of the invention can also be used in connection with the processes and methods of the invention.

In the following, the present invention will be illustrated by examples, without being limited thereto.

### EXAMPLES

### Example 1

### General Setup of Breakthrough Experiments

Cumulative residence time distribution in a column packed with non-porous beads can be obtained by so-called breakthrough experiments. For the examples of the present invention, breakthrough experiments were performed in the following three steps:
1. Flushing the column with equilibration buffer
   In the experiments of the present invention, water was used for equilibration.
2. Flushing the extra-column tubing with buffer containing the analyte acetone (with column valve on bypass)
   If not indicated otherwise, in the examples of the present invention 2% acetone was used. 2% acetone was shown to be a suitable model system for a mixture comprising the solvent/detergent mixture suitable for solvent/detergent virus-inactivating treatment according to the present invention (see Example 2). Using an acetone system instead of a mixture comprising the solvent/detergent mixture allowed for more convenient lab work. When indicated, additional experiments were performed with 10% acetone for higher sensitivity.
3. Start of breakthrough measurement by switching column valve to selected column
   The UV response was detected downstream of the column packed with non-porous beads using a UV detector. The normalized UV response represents the cumulative residence time distribution (FIG. 1A).

In the examples of the present invention, the UV detector was set to a wavelength of 280 nm, unless a mixture comprising the solvent/detergent mixture suitable for solvent/detergent virus-inactivating treatment according to the present invention was used. If the mixture comprising a solvent/detergent mixture was used, the UV detector was set to a wavelength of 300 nm, because at the wavelength with the maximum UV signal (i.e. at 280 nm), the UV detector was saturated. The breakthrough experiments were performed on the chromatographic system Aekta Avant from GE Healthcare at different superficial linear velocities ranging between 2 cm/h to 300 cm/h. For the examples of the present invention, UV spectra were processed with in-house processing scripts in Matlab® programming environment. The UV response was normalized to range from 0% to 100%. The elution volume (EV) is expressed in column volumes (CV). Elution volumes at different concentrations of flow through solution (acetone in water) were calculated (e.g. elution volume at 5% and elution volume at 50%, see FIG. 1A).

When using packed columns, low intensity peak tailing is expected to be longer than low intensity peak fronting. However, in the method for continuous virus inactivation according to the present invention, peak tailing is not as relevant as peak fronting, because viral inactivation increases over time. Thus, the narrowness of the obtained UV profiles can preferably be described by the following parameter:

$${θ}_{x} = \frac{{EV}_{x}}{{EV}_{50 \%}}$$

EV_{50%} is the mean of the residence time distribution, while EVₓ typically represents the elution volume when the signal reaches the lowest reliable detection limit (“limit of detection”, LOD). In the examples of the present invention, EV_{1%} and EV_{5%} are commonly used. It is understood that independent of the present examples, the EV_{1%} and EV_{5%} can generally be used in accordance with all embodiments of the invention. Using the setup of these examples, elution volumes down to EV_{0.03}% can be detected when 10% acetone solution is used. If θ_{X} approaches 1, the RTD is very narrow, i.e. the liquid flow through the column packed with non-porous beads approaches ideal plug flow. In contrast, if θ_{X} approaches 0, the RTD is very broad, i.e. the RTD shows severe peak fronting. Generally speaking, the closer θ_{X} is to 1, the steeper the RTD curve.

Additionally, for each breakthrough curve, the Bodenstein number was calculated by fitting function F to the normalized UV signal, where F(EV) represents integral of Gaussian peak and Bo represents the Bodenstein number, EV represents the elution volume at a given time point and EV_{50%} represents the elution volume at the mean of the RTD:

$$F ( EV ) = \frac{1}{2} ( erf ( \frac{1}{2} \sqrt{Bo} ( \frac{EV}{{EV}_{50 \%}} - 1 ) ) + 1 )$$

As is known to a person skilled in the art, also the Bodenstein number can be used as a measure of the residence time distribution. A small Bodenstein number is indicative of a broad RTD, whereas a large Bodenstein number is indicative of a narrow RTD.

### Example 2

### Comparison of Performance Between Acetone and Mixture Comprising Solvents/Detergents

Working with solvent/detergent mixtures can be hazardous, which inconveniences lab work. Thus, in order to allow for more convenient lab work, it was tested whether an acetone solution is a suitable model system for a mixture comprising the solvent/detergent mixture suitable for solvent/detergent virus-inactivating treatment according to the present invention.

Various columns were packed with glass beads. Breakthrough experiments were performed as described above (see Example 1) using a 2% acetone-in-water mixture, or a combination of process fluid buffer and process fluid buffer with addition of solvent/detergent chemicals. The ratio of EV_{5%} to EV_{50%} (θ_{5%}) and the Bodenstein numbers were calculated for each experiment.

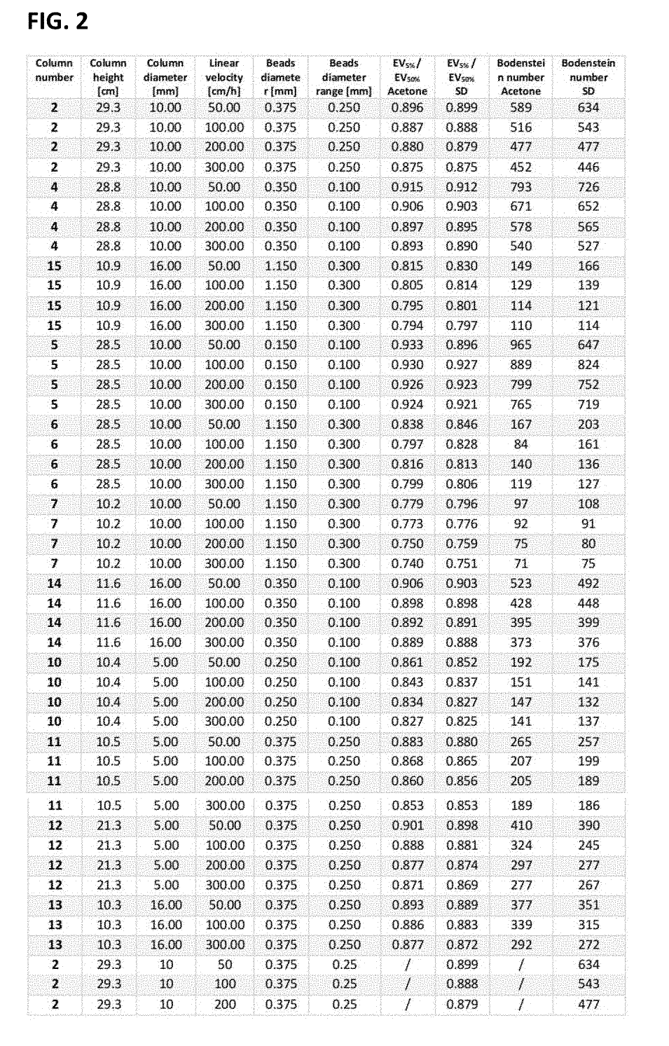

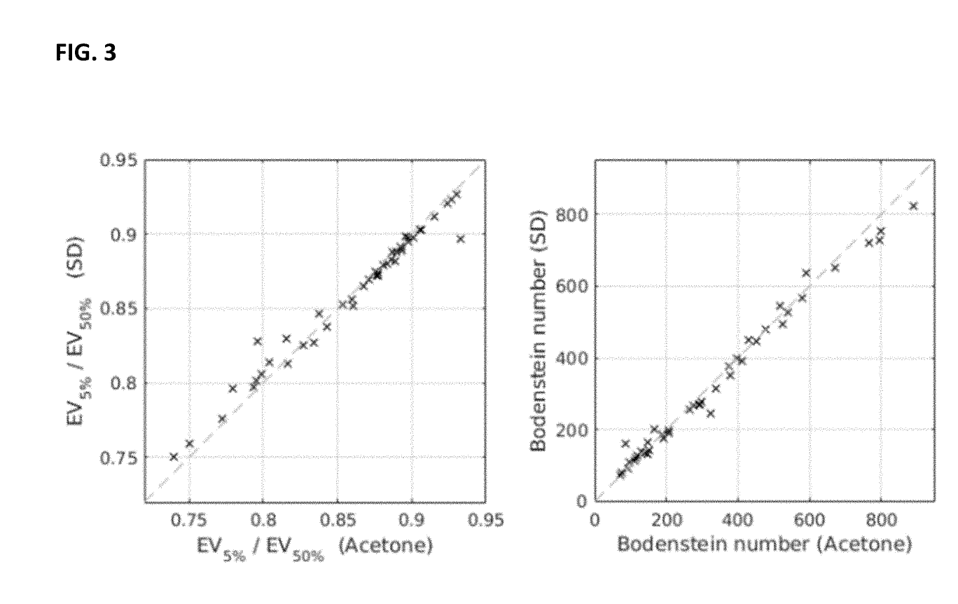

As can be seen in FIGS. 2 and 3, the θ_{5%} and the Bodenstein numbers were very similar for experiments, which differed in whether a 2% acetone-in-water mixture or a combination of process fluid buffer and process fluid buffer with solvent/detergent chemicals was used. Thus, only the combination of water and 2% acetone was used in further experiments, unless indicated otherwise.

### Example 3

### Influence of Column Parameters on Residence Time Distribution

In order to investigate the influence of various parameters of columns packed with non-porous beads on the residence time distribution, the data summarized in FIG. 2 were analyzed in regard of the column heights, column diameters, linear velocities, bead diameters and bead diameter ranges. Generally, in connection with the present invention, the terms “height” and “length” are used interchangeably and they always mean the height of the structure, e.g. the height of the packed bed.

Specifically, partial least square (PLS) analysis was performed on the data summarized in FIG. 2 for the input parameters (column height, column volume, linear velocity, mean bead diameter, bead diameter distribution) and for two output parameters (Bodenstein number and θ_{1%}). Orthogonal PLS (OPLS) regression was used to represent the influence of individual input parameters on the output.

In our case, OPLS is the same as PLS with the coordinate system rotated for more intuitive representation (Ref. 7). More particularly, the influence of individual parameters on the output can be observed from 1st OPLS component. Parameters with positive value increase the output if they are increased. Parameters with negative value decrease the output if they are decreased. If absolute value of the 1st OPLC component of a certain parameter is high, then the parameter has high influence on the output. (The 2nd OPLS component is not relevant in this case—in a simplified explanation it could be interpreted in a way that it relates to parameter variability.)

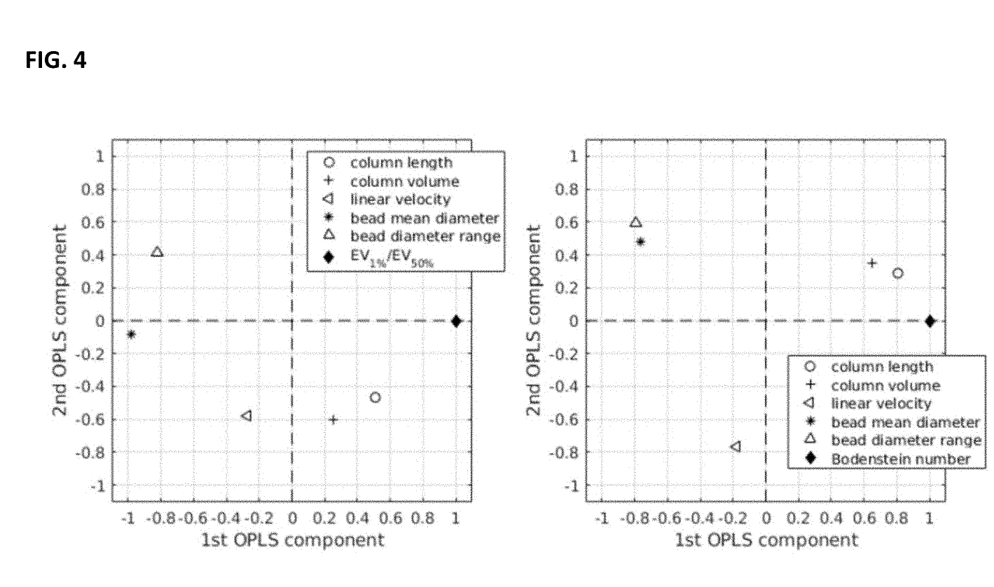

Plotting of the first two principal components revealed that the influence of bead particle dimensions is the most significant parameter in the investigated range (FIG. 4). The smaller and more uniform the beads are, the narrower is the RTD. Another significant factor was column length. Longer columns provided narrower RTD. The least influential factors were column volume and linear velocity. The latter means that for scaling up, the column diameter can be changed and/or the residence time can be increased by using decreasing linear velocities with little influence on the RTD in the tested range. However, it was observed that lower linear velocities and larger column volumes both resulted in somewhat better RTD. The above considerations were consistent for both parameters describing the RTD, i.e. the Bodenstein number and θ_{1%}.

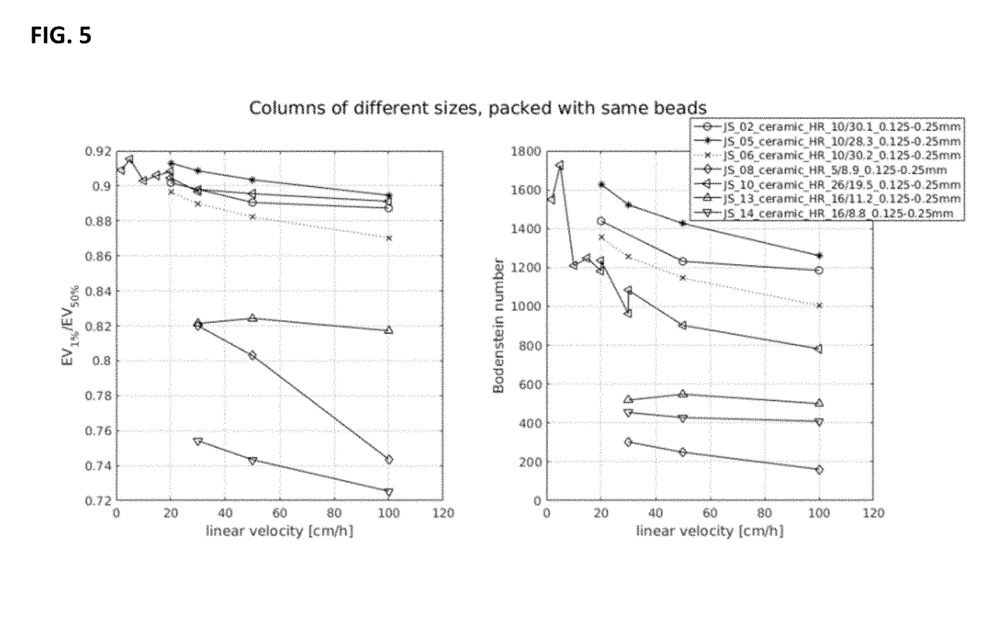

Another experiment was performed to confirm the influence of column length on RTD. Columns of different sizes were packed with ceramic beads of the same batch, and breakthrough experiments were performed at various linear velocities. Also in this experiment it was found that shorter columns have a lower θ_{1%}, i.e. a broad RTD (FIG. 5).

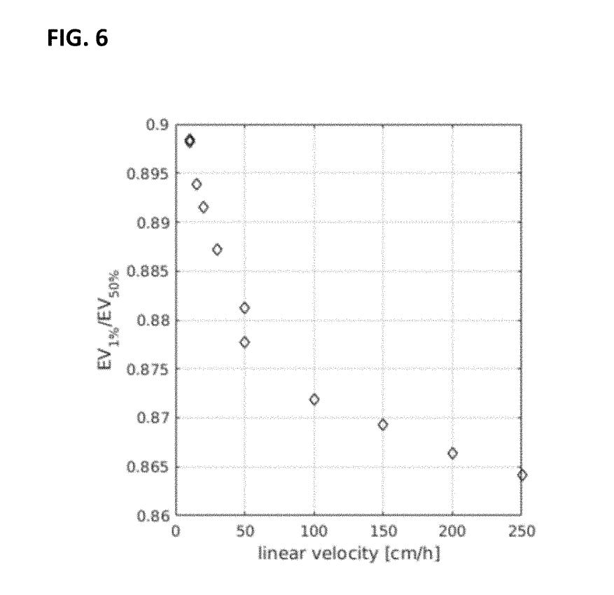

Another experiment was performed to confirm the influence of the linear flow velocity on RTD. A column suitable for the method of continuous virus inactivation according to the present invention (“MP_7_PMMA_HS_16/13.2_0.2-0.4 mm; material: PMMA plastic; diameter: 16 mm; height: 13.2 cm; bead size: 0.3 μm±0.1 μm) was packed using a vibrational column packing station. Different flow through times should result in different flow rates and thus different RTD. Thus, the RTD was assessed using the EV_{1%}/EV_{50%} (θ_{1%}), across the entire range of flow though times from 1 minute to 30 minutes. Assuming a porosity of 0.4±0.05, the superficial linear flow velocity would be in the range of between 5 cm/h and 180 cm/h. In such a range, the RTD gets wider, i.e. the θ_{1%} gets lower, towards higher velocities (FIG. 6). The range of tested linear velocities, column performance in terms of θ_{1%} drops by 4%. Notably, the column used in this experiment is short compared to what is expected to be used in a biopharmaceutical production process. As longer columns give narrower RTD (see above), in a biopharmaceutical production process, an even narrower RTD is expected.

### Example 4

### Predicting the Influence of Column Parameters and Linear Flow Velocity on RTD

Being able to accurately predict the influence of column parameters and linear flow velocity on RTD is important e.g. when scaling up the columns for integration into a biopharmaceutical production process. RTD PLS prediction was performed for all 5 input parameters (columns length, column volume, linear flow velocity, mean bead diameter and bead diameter range), and for the same input parameters except linear velocity. As can be seen in FIGS. 5 and 6, respectively, predicting the influence of the input parameters on the RTD using the PLS prediction model correlated well with the observed experimental data, regardless of whether the EV_{1%} to EV_{50%} (θ_{1%}) or Bodenstein numbers were used to evaluate the RTD. However, the θ_{1%} was more linearly correlated with the input parameters than the Bodenstein number.

### Example 5

### Influence of Column Packing on RTD

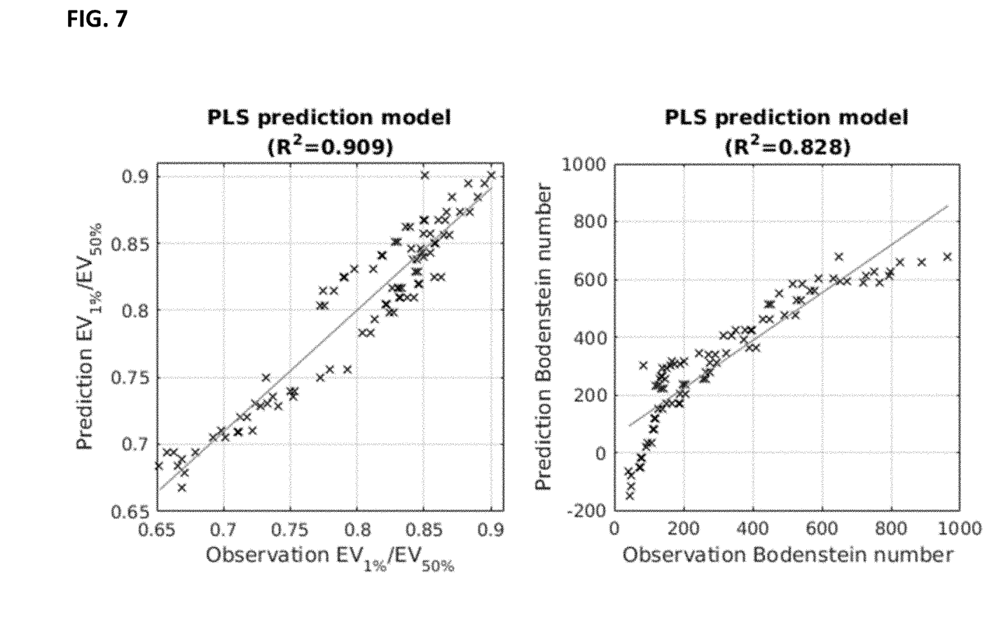

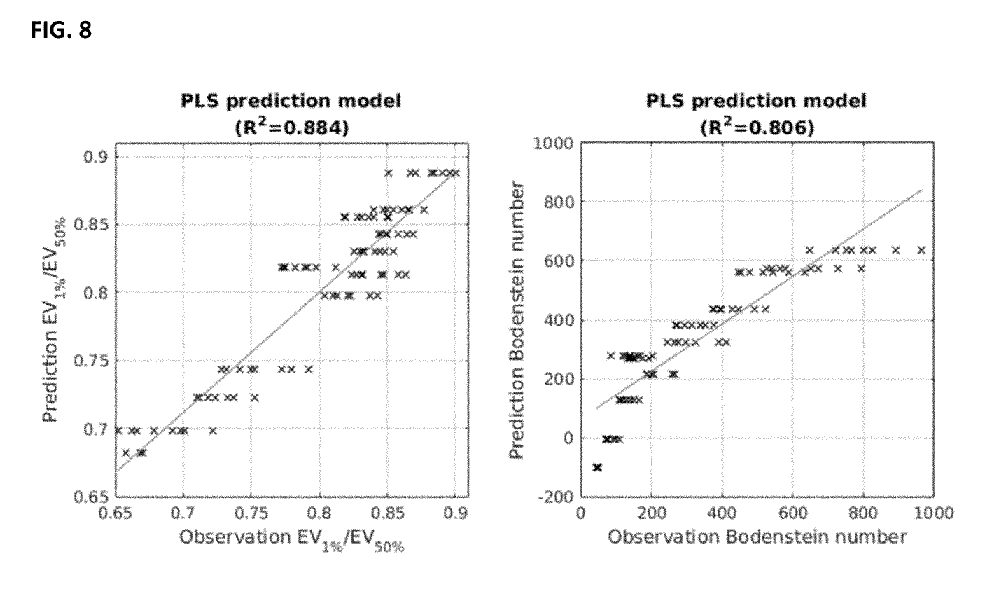

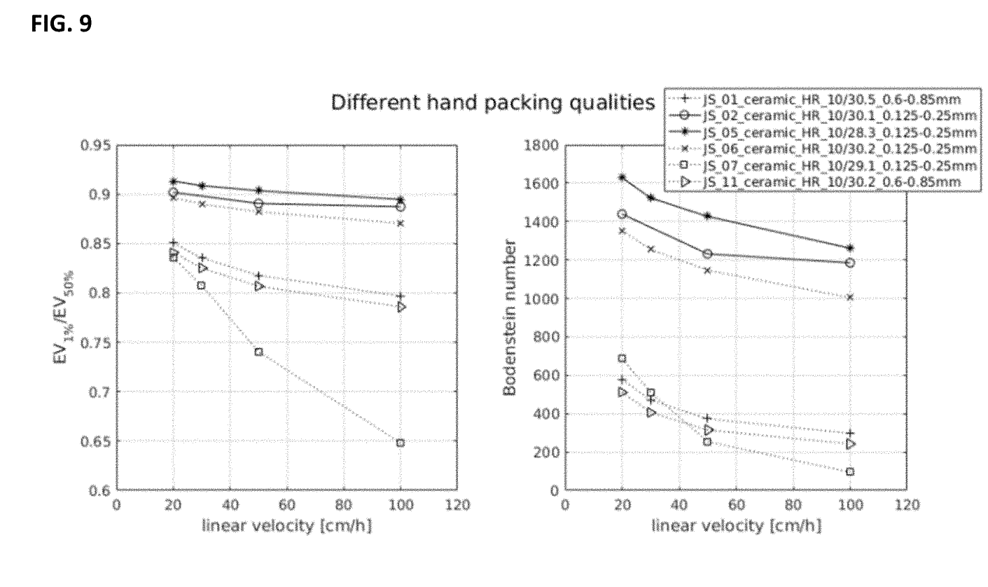

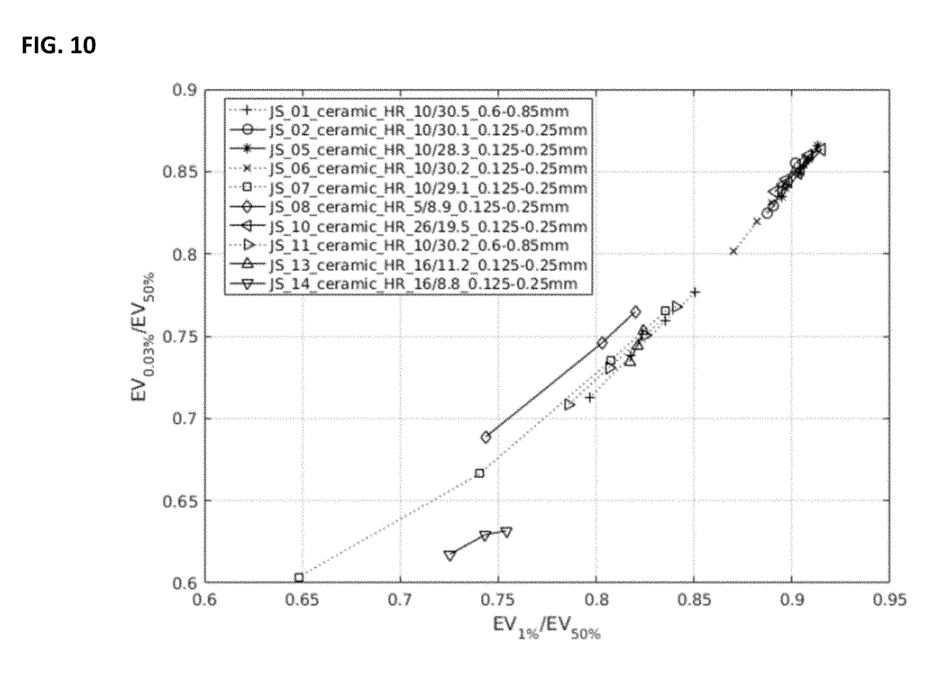

To assess the influence of column packing on the RTD, columns of the same diameter (1 cm) and similar lengths (from 28.5 cm to 30.5 cm) were hand-packed with ceramic beads. One of them (JS_07) was packed badly on purpose, i.e. it contained a lot of air bubbles after packing. At low superficial linear velocities the badly packed column performed similarly to the well packed columns with larger bead sizes (FIG. 7). However, at higher superficial linear velocities the badly packed column performed much worse, i.e. the θ_{1%} was much lower, indicating a broad RTD. These results show that the column packing quality affects the RTD. Notably, high-quality column packing can be done using e.g. a custom-built vibration station.

### Example 6

### Lowering the Detection Limit

Using 2% acetone, the limit of detection (LOD) in the breakthrough experiments is in the range of 1% of the elution volume (EV_{1%}). However, the method for continuous virus inactivation according to the present invention preferably achieves a Log10 reduction value (LRV) of at least 4. An LRV of 4 would be equivalent to a reduction from 100% infectious virus particles to 0.01% infectious virus particles. In this regard, a limit of detection of EV_{1%} is relatively large.

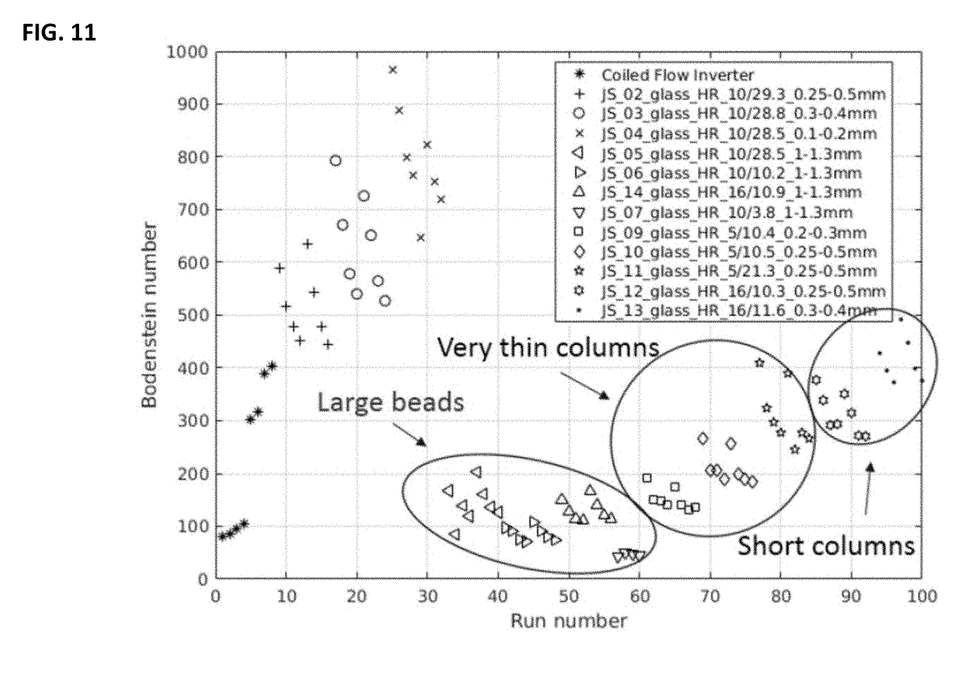

In order to achieve a lower LOD, 10% acetone was used. In this case, the LOD could be set to 0.03% at UV 280 nm. When breakthrough experiments were performed using 10% acetone and columns packed with ceramic beads, θ_{0.03%} (EV_{0.03%}/EV_{50%}) and θ_{1%} (EV_{1%}/EV_{50%}) correlated very well, especially when using well-packed columns (see FIG. 11). Thus, the use of θ_{1%} for evaluating the influence of various parameters on the RTD is justified. Notably, fluorescence experiments could be used to obtain even lower limits of detection.

### Example 7

### Comparison to Known Methods

In the known methods, coiled flow inverters (CFI) have been used to achieve a narrow RTD. However, the scalability of the packed bed of non-porous beads according to the present is much better than the scalability of CFIs, because for the packed bed of non-porous beads, the RTD gets narrower when using longer beds, and the bed is not very sensitive to changes in flow rates. In contrast, CFIs are only proven to work with tube diameters of 2-3 mm, and scale-up capabilities are questionable due to non-ideal fluid dynamics. Moreover, CFIs are limited to a single flow rate for each given design.

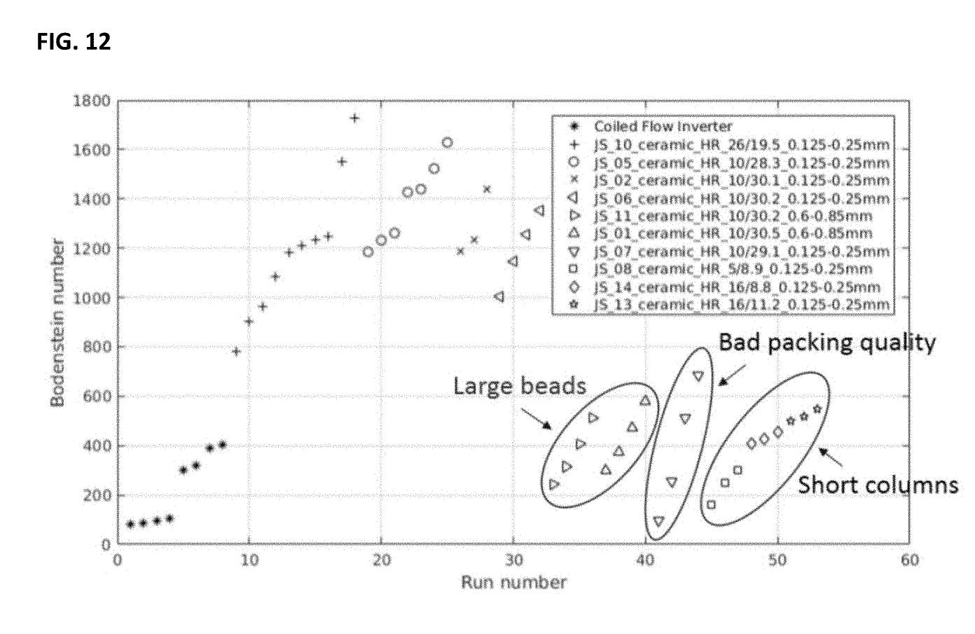

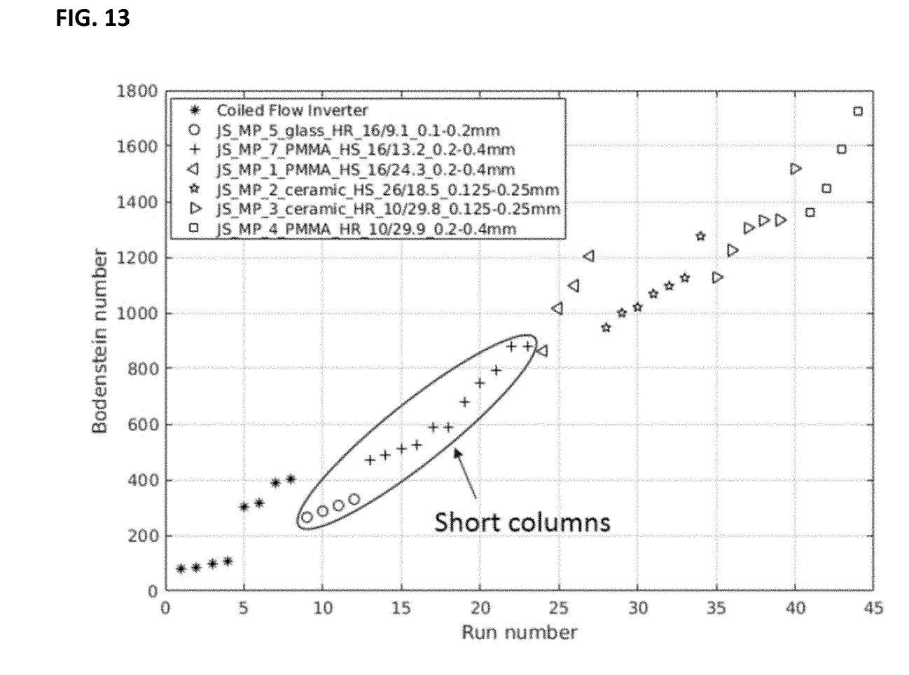

In order to compare the RTD of CFIs with the RTD of columns packed with non-porous beads according to the present, the Bodenstein numbers obtained with CFIs and published in Klutz et al. (Ref. 2) were compared to the Bodenstein numbers achieved by the packed columns of the present invention. Strikingly, the Bodenstein numbers of columns with diameters of more than 5 mm, lengths of more than 10 cm, and with beads that were smaller than 600 μm in diameter were higher than the Bodenstein numbers of the CFIs described in the known methods, regardless of whether glass beads (FIG. 11), ceramic beads (FIG. 12), or PMMA plastic beads (FIG. 13) were used.

### Example 8

### Residence Time Distribution for Columns of the Invention and Comparative Columns at Different Column Sizes

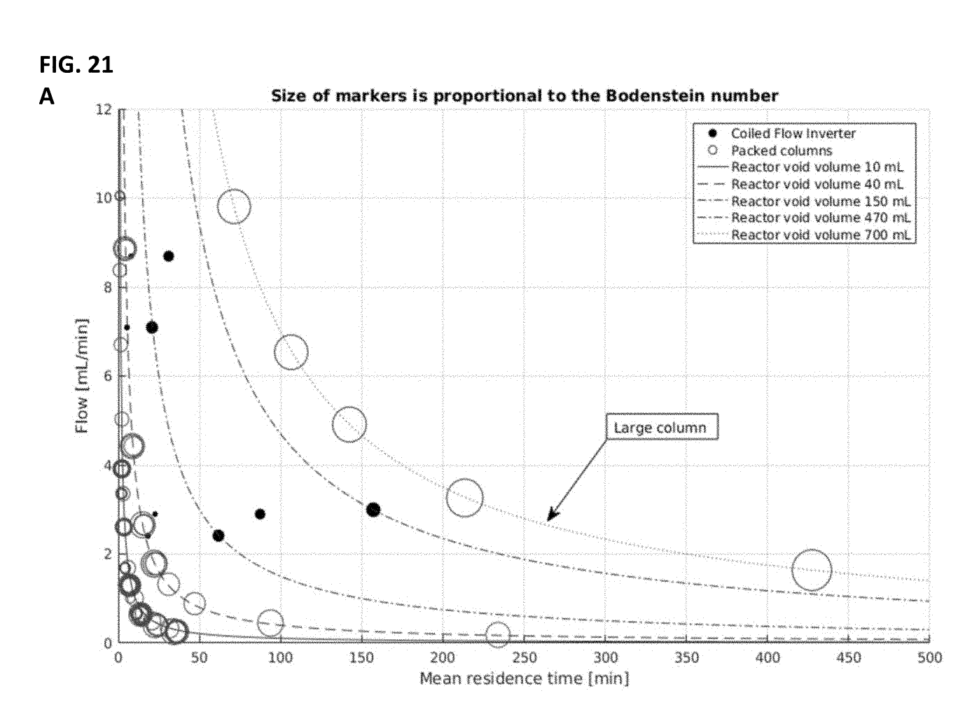

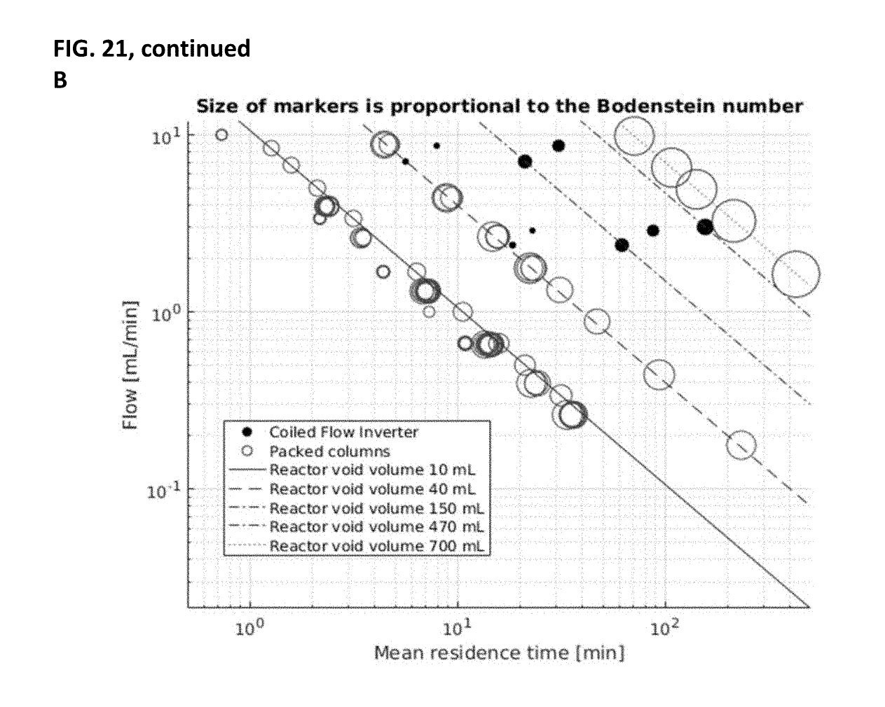

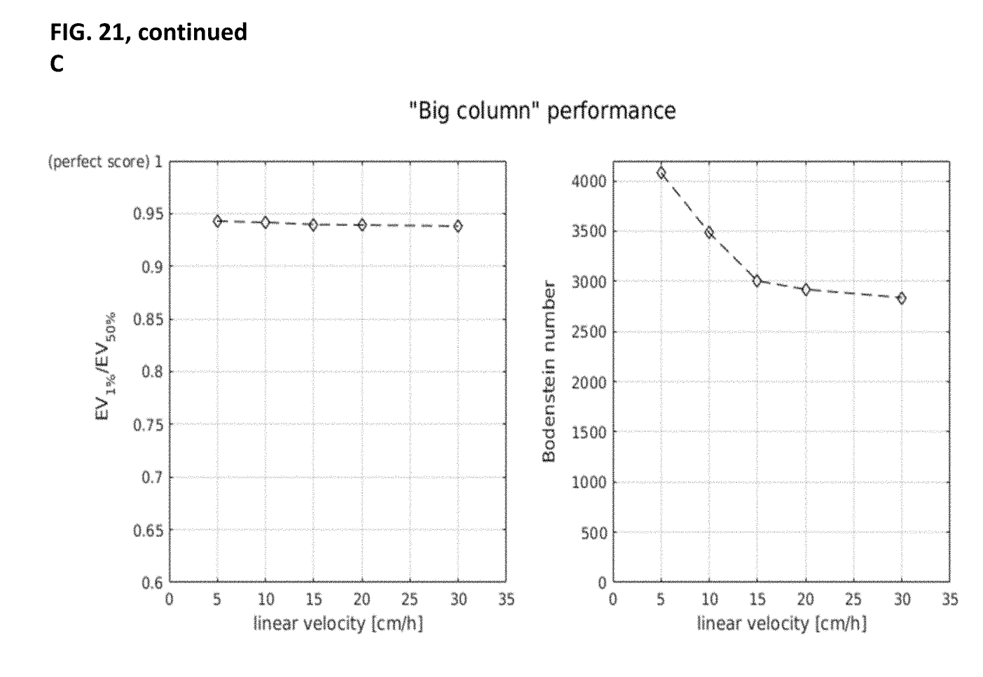

Next the inventors investigated the influence of the size of the columns on the residence time distribution and also compared the columns of the invention to converted flow inverter (CFI) columns. The results are shown in FIG. 21. In FIG. 21 A, each circle represents an experiment. The size of the circle is proportional to the Bodenstein number. Thus, larger the circle means larger the Bodenstein number, which means the closer the system is to the ideal plug flow. On the x-axis, the mean residence time (or flow through time) is shown, and on the y-axis is the flowrate. Empty circles represent experiments with packed columns according to the invention, and full circles represent data from a comparative coiled flow inverter (CFI). Dashed lines are representing the trajectory one would obtain while using a single reactor (or multiple reactors with same void volumes) at different flowrates. The purpose of this plot is to put the comparison in perspective regarding the used flowrate and reactor size, as it would be inappropriate to compare the Bodenstein numbers between two methods performed at very different flowrates or at different scale.

Although the inventors had already demonstrated than the columns according to the invention have smaller void volumes than CFI setups and that the residence time distribution (RTD) gets narrower with scaling up the column, the inventors also performed an additional direct comparison in form of a plot. In the plot of FIG. 21 A, also results from one large packed column are shown. While the other columns had smaller void volumes than most of the CFI setups presented, this large column was larger than all of the CFI setups. The large column substantially outperforms all smaller (lab scale) columns as well as all CFI setups (note that the large clear circles belong to the large column).

FIG. 21 B is the same as FIG. 21 A, except that the scales are in logarithmic form. Thus experiments with the same void volume (same reactor) lie on a straight line.

The experiments, which were performed for the large column, are depicted in more detail in FIG. 21 C. In particular, a column (GE Healthcare XK 50/100) with diameter of 5 cm and length of 89 cm was packed with ceramic beads with a diameter of between 125 μm and 250 μm. The total volume of the packed column was 1.75 L, and the void volume was 0.7 L. The column was packed using a vibration column packing station. The purpose was to confirm the trend of a narrower residence time distribution (RTD) with scaling up the column, as well as to demonstrate narrow RTD also for a column larger than the comparative Coiled flow invertor (CFI) reactors.

Experiments were performed with a superficial linear velocity of 5 cm/h, 10 cm/h, 15 cm/h, 20 cm/h and 30 cm/h. The range of volumetric flowrates was still broader on the upper and lower limit than for the flowrates used in the CFI reactors.

The large column produced a very narrow RTD, as expected in accordance with the invention (FIG. 21 C). In comparison, a smaller column (d=26 mm, I=19.5 cm) packed with the same batch of beads achieved an EV_{1%}/EV_{50%} score in the range of 0.88-0.92 and a Bodenstein number in range of 800-1800.

### Example 9

### Exemplary Embodiment of the Device for Preparation of a Biopharmaceutical Drug

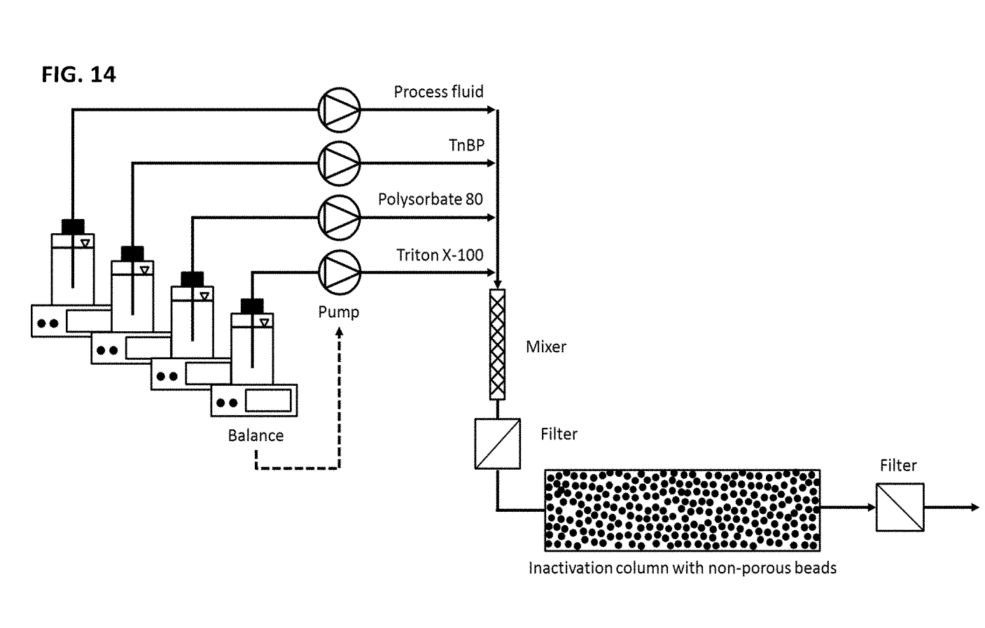

An exemplary embodiment of the device for preparation of a biopharmaceutical drug is shown in FIG. 14. The process fluid is mixed with stock solutions of the individual solvent/detergent chemicals. Balances provide feed-back control to ensure correct flow rates of all components to achieve the desired final concentrations. Inline mixers homogenize the solutions. The homogenous solution enters the inactivation column after being passed through an absolute filter (e.g. a 0.2 μm filter) to remove particulates.

### Example 10

### Mathematical Approach for Estimating Virus Inactivation

Two approaches for claiming viral inactivation were suggested for continuous setups by Klutz et al. (Ref. 3). The first approach is based on the peak start detection (with limit of detection set to 0.5% of breakthrough), where the peak start elution time should be the same as the viral inactivation time in the corresponding batch reactor. 99.5% of process fluid would have longer incubation time than in batch process and thus, the log reduction value (LRV) of the continuous setup is expected to be higher than the batch operation.

$$LRV = {log}_{10} \frac{Virus titer before inactivation}{Virus titer after inactivation}$$

The second approach is assuming an exponential nature of viral inactivation (which is confirmed by experimental batch inactivation kinetic results). Effective LRV for second approach is defined as average LRV weighted by residence time distribution (RTD). This approach then allows shorter residence times in the reactor because as the aim is to reach the same LRV as in batch operation. However, the suggestions were not accompanied by calculations.

The onset of the RTD peak is critical part, as viruses eluting early in the very onset of the peak have relatively short incubation time. The study of the onset of the peak was not considered in the methods known in the art.

In the packed-column based method of the present invention, we therefore suggest to divide the breakthrough profile into two sections—before and after we are able to detect the very onset of the breakthrough curve. This occurs once the signal surpasses out lower limit of detection (LOD). The profile before the detection is not known. The breakthrough profile represents cumulative residence time distribution, whereas a pulse injection profile would represent a normal residence time distribution. Thus the elution time at which the breakthrough curve rises over LOD (LOD time, tᵢₙᵢₜ) is the time when the φᵢₙᵢₜ share of the RTD peak would elute:

φᵢₙᵢₜ=LOD(UV signal)/max(UV signal)

If one assumes that there is no viral inactivation in the initial part before limit of detection (φᵢₙᵢₜ), then the limit of detection should be very low in order to achieve required LRV.

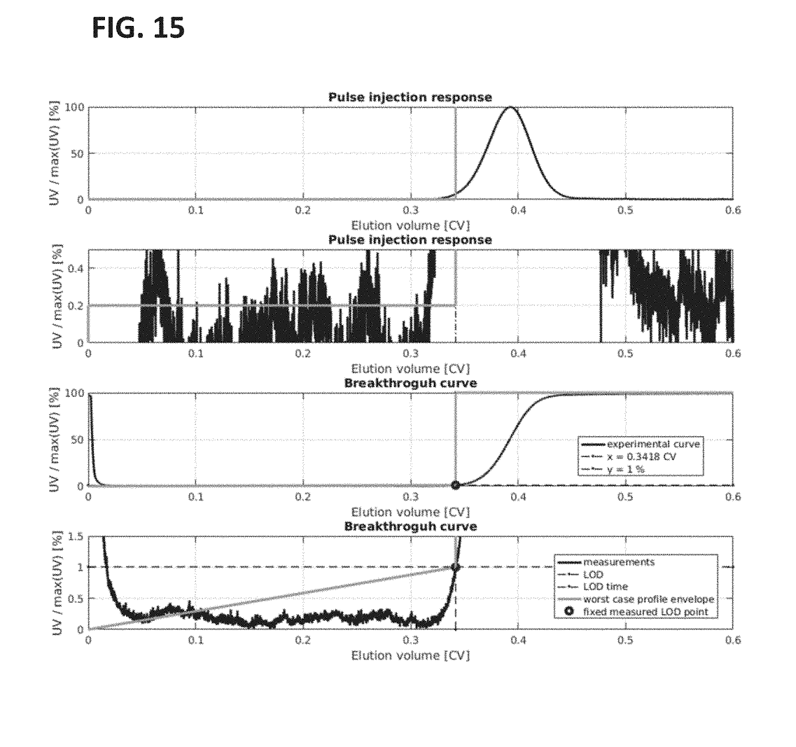

In our column there is no binding to and no pores in the particles, thus the RTD is expected to have only one peak. If there is only one peak, the worst case theoretical scenario with the lowest average residence time, while assuming a single peak profile, would be a constant sample concentration before the elution of detectable elution peak i.e. extreme peak fronting (FIG. 15).

Assuming an exponential nature of viral inactivation and the worst case peak fronting scenario described above, the virus reduction ratio for φᵢₙᵢₜ can be calculated (noted by RVᵢₙᵢₜ).:

$${RV}_{init} = \frac{1 - {exp}_{10} ( - k {t}_{init} )}{k {t}_{init} ln ( 10 )}$$

The coefficient for exponential viral inactivation decay (k) can be calculated from the required batch viral inactivation incubation time (t₀) and the corresponding lower limit for viral inactivation (RVₘᵢₙ=reduction value).

RVₘᵢₙ=exp₁₀(−k t₀)

The incubation time of material eluted after LOD is set to the LOD time. The joined reduction value (RVₜₒₜₐₗ) is calculated from both contributions and should be equal to the RVₘᵢₙ.

$${RV}_{total} = {ϕ}_{init} * {RV}_{init} + ( 1 - {ϕ}_{init} ) * {exp}_{10} ( - k {t}_{init} ) = {ϕ}_{init} * \frac{1 - {exp}_{10} ( - k {t}_{init} )}{k {t}_{init} ln ( 10 )} + ( 1 - {ϕ}_{init} ) * {exp}_{10} ( - k {t}_{init} ) = {RV}_{min}$$

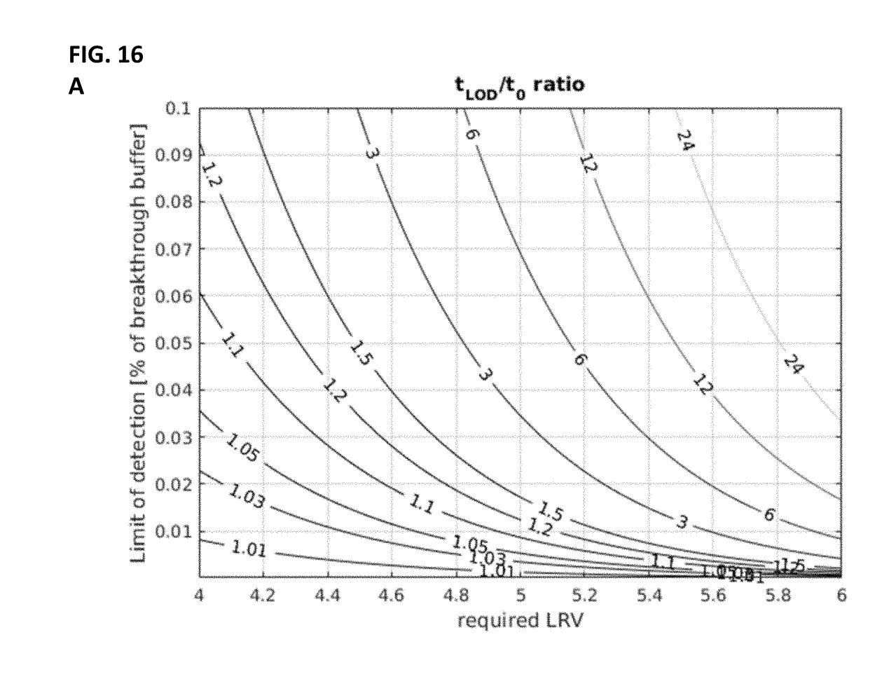

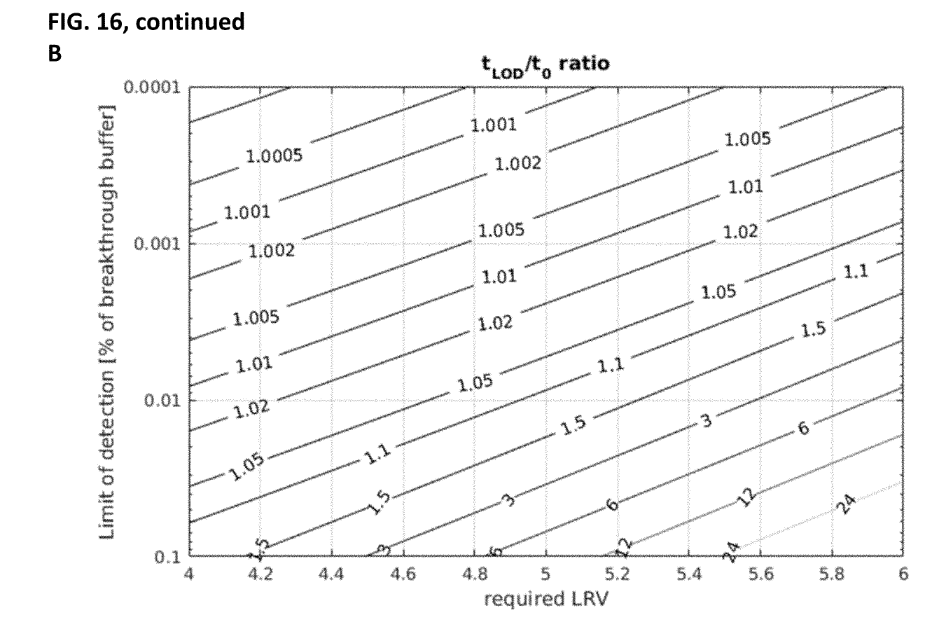

From the upper equation the required LOD time for required RVₘᵢₙ and for the given LOD can be calculated (FIG. 16).

Step-by-step example:
1) It was shown above that LOD<0.03% is achievable by use of 10% Acetone and UV detector. Thus in this example the inventors used φᵢₙᵢₜ=0.03%, required LRV 4 logs and a batch incubation time (t₀) of 1 hour. The required LOD time can be estimated from the plot in FIG. 16. A precise value can be obtained by numerical solving of the last equation. The value estimated from the plot is 1.05. tᵢₙᵢₜ=1.05; t₀=1.05*60=63 min.
2) The inventors have also shown above that for LOD<0.03% the ratio between LOD time and mean residence time above 0.8 is achievable (EV_{0.03%})/(EV_{50%})=0.8). Thus the mean residence time (tₘₑₐₙ) is: tₘₑₐₙ=t_{LOD}/0.8=79 min
3) A typical porosity is ≈0.4. It depends on particle size distribution. For this example we can take porosity is =0.4 and desired method ϕ_{throughput}=1 L/hour. In this case the total column volume

$$CV = \frac{{t}_{mean} * {∅}_{throughput}}{ɛ} = 3.3 L$$

### Example 11

### Virus Inactivation

An exemplary virus inactivation according to the invention can be carried out as follows. In the example below, the entire setup as well as all solutions is at room temperature. The entire inactivation process is continuously operated.

A buffered solution containing a proteinaceous product (20 mM MES, 10 mM CaCl2, 0.1% Polysorbate 80, 500 mM NaCl, pH 6.35) is continuously mixed with a stock solution of solvent-detergent chemicals: Tri-n-butyl-phosphate, Triton X-100 and Polysorbate 80 (mass percentages of the three chemicals in the stock solution: 17.47%:63.25%:19.28%). A dynamic inline mixer is used for mixing the two solutions. The volumetric flow rates of the two streams are 0.161 mL/min and 10.0 mL/min for the solvent-detergent stock and for the product-containing stream, respectively. The resulting homogeneous mixture passes an inline filter to remove any particulates. The solution is then fed directly into the inactivation column packed with non-porous beads and a column volume of 2134 mL. The column height is 27.2 cm and the column diameter is 10 cm. The column is a column equilibrated with buffer (20 mM MES, 10 mM CaCl2, 0.1% Polysorbate 80, 500 mM NaCl, pH 6.35) with the same SD concentration as are present in the mixture of product solution and SD chemical stock solution.

The outflow of the virus inactivation column is filtered through a filter, inline diluted 1:4.5 with a buffer solution (50 mM Tris, 5mM CaCl2, 0.1% Polysorbate 80) and loaded onto a wide-bore anion exchange column.

### Example 12

### Virus Inactivation (X-MuLV at 5% SID)

Below an experimental example for continuous viral inactivation (CVI) is described, wherein the solvent/detergent (S/D) process was used, and wherein continuous viral inactivation was compared against the industry-standard S/D batch incubation.

The experiments were performed accordingly with the industry-relevant guidelines, such as, but not limited to, the ICH Q5A(R1) 1999 guideline, ICH CPMP/BWP/268/95 1996 guideline and the EMEA CHMP/BWP/398498/2005 2009 guideline.

The virus titer was determined by the 50% Tissue Culture Infective Dose (TCID₅₀) method. The limit of detection (LOD) and lack of sample interference was assessed for the TCID₅₀ by a person skilled in the art.

The continuous virus inactivation reactor (CVIR) was used for viral inactivation in continuous operation mode. The reactor volume (V_{R}) is equivalent to EV_{1%} and was assessed by residence time analysis. The reactor was designed and operated to deliver an incubation time of 30 and 60 min. The pre-CVIR volume is small in comparison with the CVIR volume and was not considered in the residence time distribution analysis.

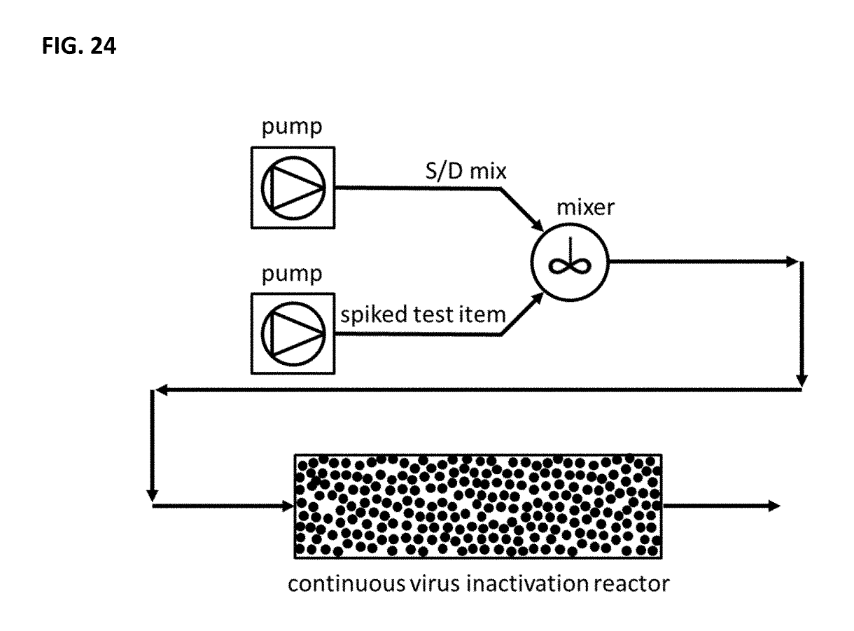

The setup used for the continuous virus inactivation is depicted in FIG. 24. In this example two pumps were used to pump the test item (a surrogate for the process intermediate) and the S/D reagent, the two streams converged at the inline mixer, where they were homogenized. Once homogeneous, a single stream was further pumped through the CVIR, where the virus inactivation took place continuously.

The CVIR was a cylindrical tube packed with poly(methyl methacrylate) (PM MA) spherical non-porous beads with diameters ranging from 200 to 400 μm with a mean diameter of 300 μm. The reactor was packed using a custom-built vibration-assisted packing station. The packing resulted in a reactor with a packed height of 132 mm and a void volume of 10.66±0.06 mL. The Bodenstein number at 10 cm/h was >875. The EV1/EV50 at 10 cm/h was 0.882, hence the CVIR volume was calculated to be 9.40±0.15 mL.

The flow rate at the CVIR's inlet and outlet was such that the incubation time was 30 and 60 min, which resulted in linear velocities inside the CVIR of 4.68 and 9.35 cm/h, respectively.

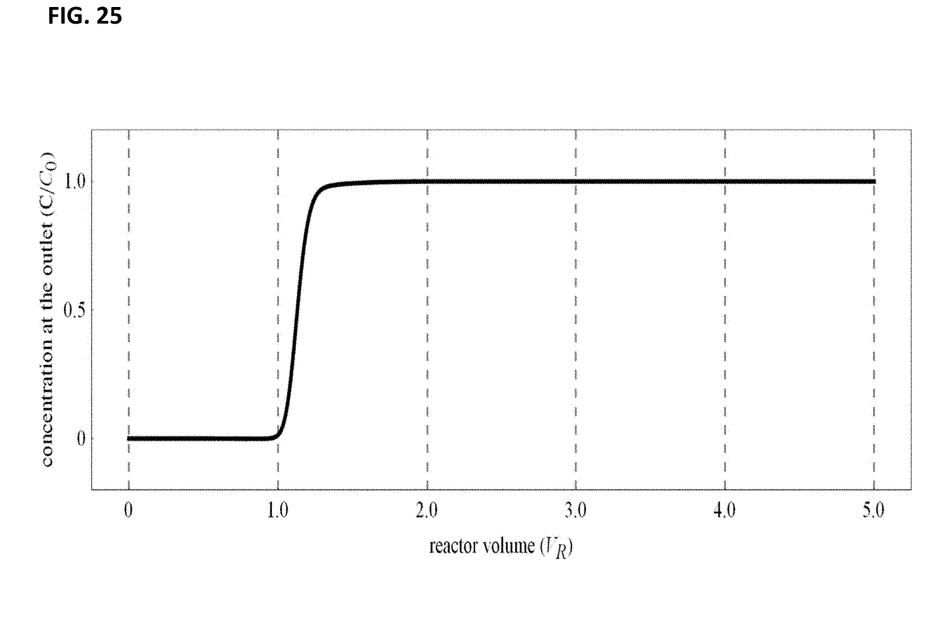

The process achieved steady state before 2 V_{R} of operation and at 2 V_{R} the system was already in steady state. Once the S/D components' concentration at the outlet reached the same concentration as at the inlet, the system had achieved the steady state, as shown in FIG. 25. The CVI process showed a latency phase and a delayed onset of the steady state due to the displacement of the liquid phase inside the CVIR, which did not contain any of the S/D components, hence no or limited virus inactivation occurred.

The test item consisted of an industry-relevant buffer with human serum albumin as an example of a biopharmaceutical drug. The test item in the present example reproduces key properties (pH, conductivity, total protein) of a process intermediate in a process for the production of a biopharmaceutical drug. The test item was spiked beforehand with X-MuLV by a person skilled in the art accordingly with the relevant guidelines.

The S/D reagent of this non-limiting example was a mixture of a solvent and detergents with virus-inactivating effect. In the present example Triton X-100 (TX-100), Polysorbate 80 (PS80) and Tri-n-butyl-phosphate (TnBP) were used. The S/D reagent was diluted at the mixer to achieve the target concentration of 0.0473% (w/w) TX-100, 0.0144% (w/w) PS80 and 0.0131% (w/w) TnBP during the CVI incubation.

A sample of the spiked test item was drawn before the starting the CVI experiment to establish the initial virus titer. The stream at the CVIR's outlet was sampled at 1, 2, 3, 4 and 5 V_{R}. The outlet samples were immediately diluted 20-fold to stop the virus inactivation process and immediately titrated for virus titer in order to establish the titer after the CVI process. A sample of the spiked test item was drawn after completing the CVI experiment to serve as a hold control (HC).

The virus inactivation was measured by calculating the logarithmic reduction value (LRV) as in equation 1 below. Equation 1 reflects the specific nature of the continuous operation and the fact that in this example there are two streams being pumped through the CVIR and only one exiting the CVIR. Therefore the virus input per unit of time before virus inactivation and the virus output per unit of time after virus inactivation are calculated based on the stream's virus titer and its respective volumetric flow rate. The titerₒᵤₜₗₑₜ was corrected for the virus inactivation-stopping dilution.

$$LRV = {log}_{10} ( \frac{{titer}_{spiked test item} \times flow {rate}_{spike test item}}{{titer}_{outlet} \times flow {rate}_{outlet}} ) \qquad ( 1 )$$

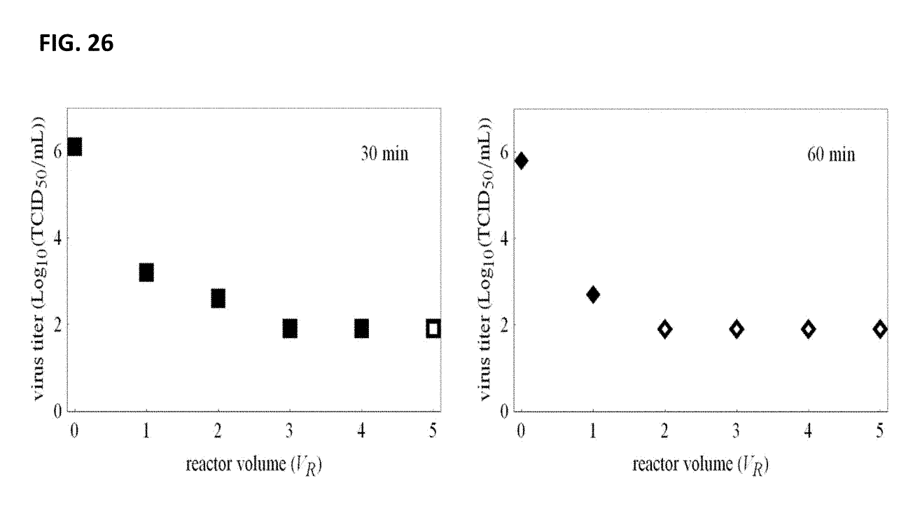

In FIG. 26 the X-MuLV titer profile after 30- and 60-min incubation CVI process is depicted. Once the operation reached steady state the X-MuLV was reduced from ≥6.3E+5 TCID₅₀/mL at the inlet of the CVIR to ≤4.0E+2 TCID50/mL at the outlet of the CVIR for the 30 min incubation time and reduced to ≤8.0E+1 TCID₅₀/mL for the 60 min incubation time. Before reaching the steady state, i. e. at 1 V_{R}, the X-MuLV titer of 2.5E+2 TCID₅₀/mL was higher than those achieved in the steady state phase. This difference can be explained by the S/D components' concentration below the target concentration at 1 V_{R} as described above.

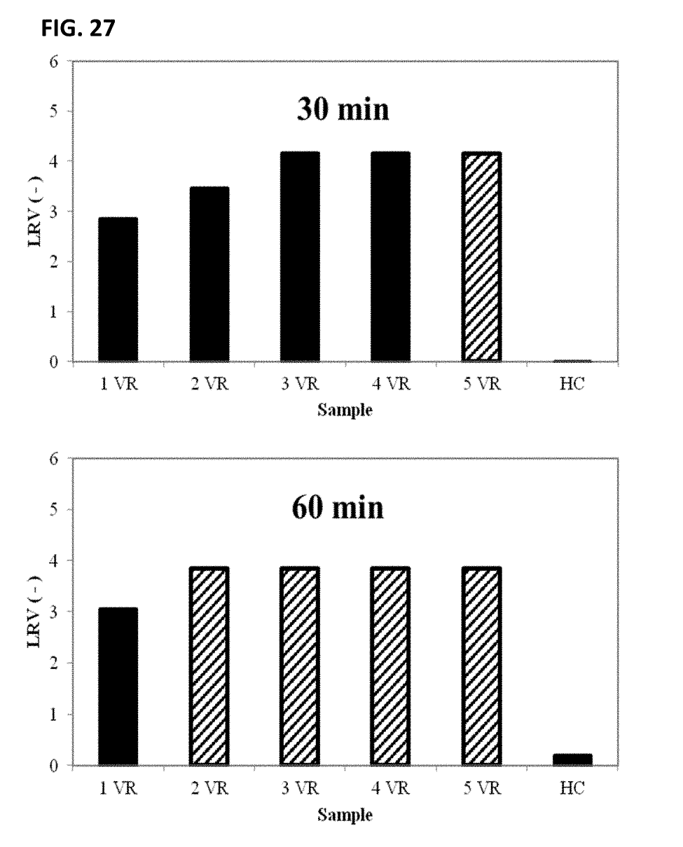

Once in steady state (from 2 V_{R} onwards) an LRV of ≥3.5 was observed for 30 min incubation time and an LRV of >3.9 for 60 min incubation time (FIG. 27). For the 30- and 60-min incubation the hold control showed a virus loss below 1 log₁₀—the minimum difference value to be considered significant by a person skilled in the art and accepted in the industry-relevant guidelines.

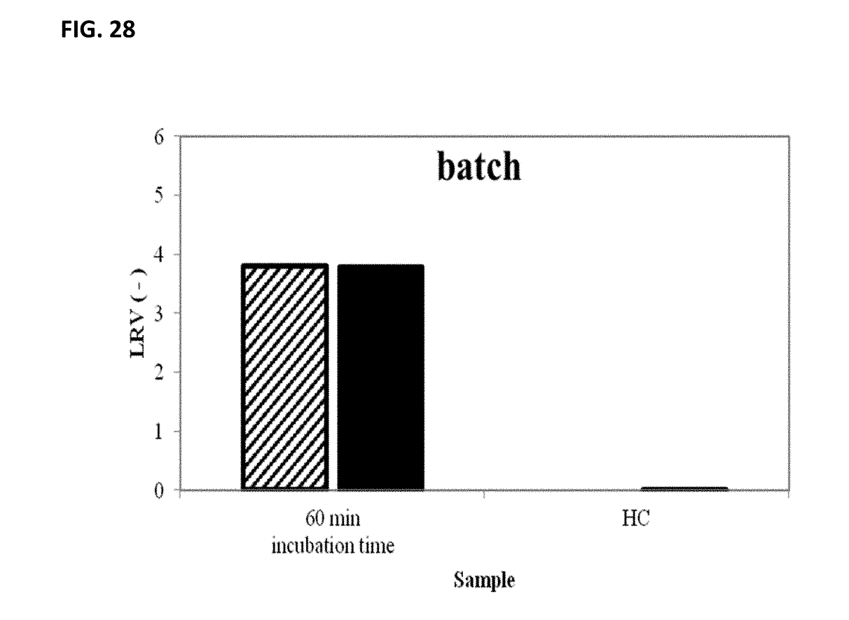

Batch experiments were performed by a person skilled in the art following the industry-relevant guidelines for comparison. The data resulting from the traditional batch (shown in FIG. 28) showed a LRV ≥3.8, which is comparable with those obtained in the CVIR in the continuous operation mode. This direct comparison showed that the continuous virus inactivation using the CVIR is as effective as the traditional batch operation.

This indicates that continuous virus inactivation according to the invention is highly advantageous, because it is as effective as the ideal inactivation conditions of viral inactivation in the batch mode (e.g. essentially equal residence time for all parts of the mixture due to a narrow residence time distribution, leading to efficient viral inactivation in all parts of the mixture), while providing the additional advantage that it can be carried out continuously.

It will be understood by a person skilled in the art that the conditions used in the viral inactivation example are not limiting to the scope of the invention. For example, while poly(methyl methacrylate) (PMMA) spherical non-porous beads with diameters ranging from 200 to 400 μm with a mean diameter of 300 μm were used as an example, any structure having multiple interconnected channels, for example any column packed with non-porous beads, can be used in accordance with the invention. Similarly, while a cylindrical tube packed using a custom-built vibration-assisted packing station was used as the CVIR having a packed height of 132 mm, a void volume of 10.66±0.06 mL and a CVIR volume of 9.40±0.15 mL, any other CVIR as defined by the present invention can be used.

### Example 13

### Virus Inactivation (BVDV at 5% SID)

Below an experimental example for continuous viral inactivation (CVI) is described, wherein the solvent/detergent (S/D) process was used, and wherein continuous viral inactivation was compared against the industry-standard S/D batch incubation.

The experiments were performed accordingly with the industry-relevant guidelines, such as, but not limited to, the ICH Q5A(R1) 1999 guideline, ICH CPMP/BWP/268/95 1996 guideline and the EMEA CHMP/BWP/398498/2005 2009 guideline.

The virus titer was determined by the 50% Tissue Culture Infective Dose (TCID₅₀) method. The limit of detection (LOD) and lack of sample interference was assessed for the TCID₅₀ by a person skilled in the art.

The continuous virus inactivation reactor (CVIR) was used for viral inactivation in continuous operation mode. The reactor volume (V_{R}) is equivalent to EV_{1%} and was assessed by residence time analysis. The reactor was designed and operated to deliver an incubation time of 30 and 60 min. The pre-CVIR volume is small in comparison with the CVIR volume and was not considered in the residence time distribution analysis.

The setup used for the continuous virus inactivation is depicted in FIG. 24 of the previous example. In this example two pumps were used to pump the test item (a surrogate for the process intermediate) and the S/D reagent, the two streams converge at the inline mixer, where they are homogenized. Once homogeneous, a single stream was further pumped through the CVIR, where the virus inactivation took place continuously.

The CVIR was a cylindrical tube packed with poly(methyl methacrylate) (PM MA) spherical non-porous beads with diameters ranging from 200 to 400 μm with a mean diameter of 300 μm. The reactor was packed using a custom-built vibration-assisted packing station. The packing resulted in a reactor with a packed height of 132 mm and a void volume of 10.66±0.06 mL. The Bodenstein number at 10 cm/h was >875. The EV1/EV50 at 10 cm/h was 0.882, hence the CVIR volume was calculated to be 9.40±0.15 mL.

The flow rate at the CVIR's inlet and outlet was such that the incubation time was 30 and 60 min, which resulted in linear velocities inside the CVIR of 4.68 and 9.35 cm/h, respectively.

The process achieved steady state before 2 reactor volumes (V_{R}) of operation and at 2 V_{R} the system was already in steady state. Once the S/D components' concentration at the outlet reached the same concentration as at the inlet, the system had achieved the steady state, as shown in FIG. 25 of the previous example. The CVI process showed a latency phase and a delayed onset of the steady state due to the displacement of the liquid phase inside the CVIR, which did not contain any of the S/D components, hence no or limited virus inactivation occurred.

The test item consisted of an industry-relevant buffer with human serum albumin as an example of a biopharmaceutical drug. The test item in the present example reproduces key properties (pH, conductivity, total protein) of a process intermediate in a process for the production of a biopharmaceutical drug. The spiked test item was spiked beforehand with BVDV by a person skilled in the art accordingly with the relevant guidelines.

The S/D reagent was a mixture of a solvent and detergents with virus-inactivating effect. In the present example Triton X-100 (TX-100), Polysorbate 80 (PS80) and Tri-n-butyl-phosphate (TnBP) were used. The S/D reagent was diluted at the mixer to achieve the target concentration of 0.0473% (w/w) TX-100, 0.0144% (w/w) PS80 and 0.0131% (w/w) TnBP during the CVI incubation.

A sample of the spiked test item was drawn before the starting the CVI experiment to establish the initial virus titer. The stream at the CVIR's outlet was sampled at 1, 2, 3, 4 and 5 V_{R}. The outlet samples were immediately diluted 20-fold to stop the virus inactivation process and immediately titrated for virus titer in order to establish the titer after the CVI process. A sample of the spiked test item was drawn after completing the CVI experiment to serve as a hold control (HC).

The virus inactivation was measured by calculating the logarithmic reduction value (LRV) as in equation 1 of the previous example. The dilution factor serves to account for the dilution of the spiked test item stream with the S/D reagent stream. The titerₒᵤₜₗₑₜ was corrected for the virus inactivation-stopping dilution.

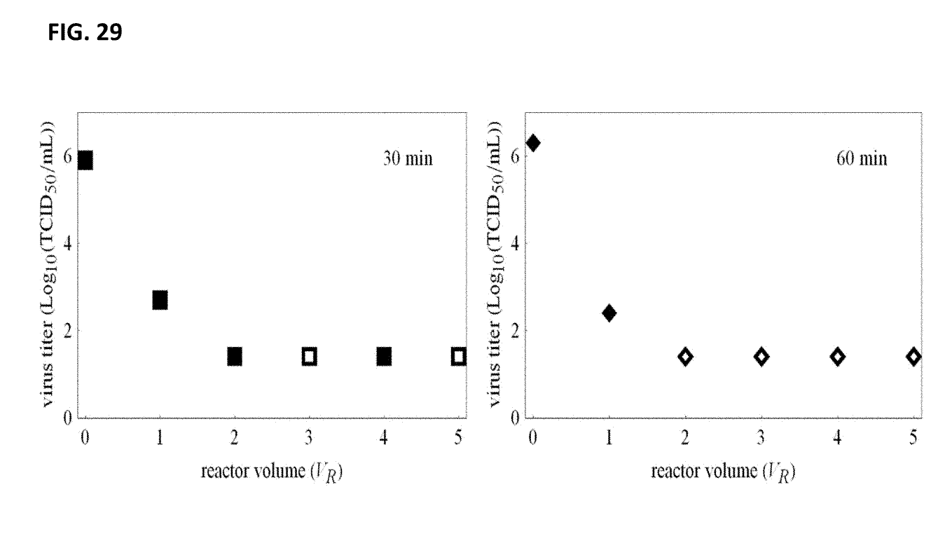

In FIG. 29 it is depicted the BVDV titer profile after 30- and 60-min incubation CVI process. Once the operation reached steady state the BVDV was reduced from 7.9E+5 TCID₅₀/mL at the inlet of the CVIR to ≤2.5E+2 TCID₅₀/mL at the outlet of the CVIR regardless of the incubation time. Before reaching the steady state, i. e. at 1 V_{R}, the BVDV titer of ≤5.0E+2 TCID₅₀/mL was higher than those achieved in the steady state phase. This difference can be explained by the S/D components' concentration below the target concentration at 1 V_{R} as described above.

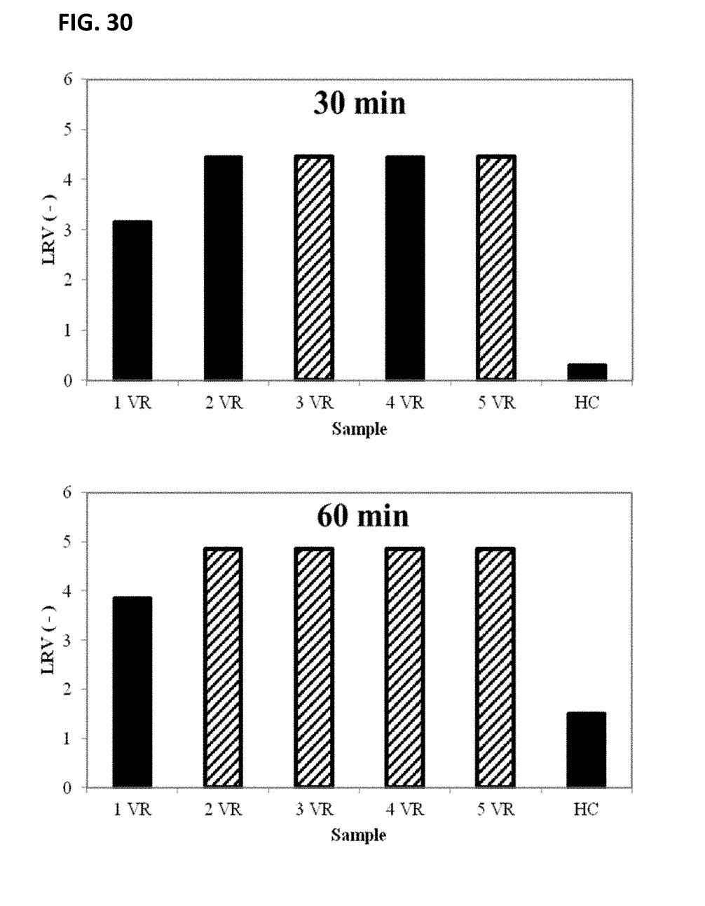

Once in steady state (from 2 V_{R} onwards) an LRV of ≥4.5 was observed for 30 min incubation time and an LRV of ≥4.9 for 60 min incubation time (FIG. 30). For the 30 min incubation the hold control showed a virus loss below 1 log₁₀—the minimum difference value to be considered significant by a person skilled in the art and accepted in the industry-relevant guidelines). For the 60 min incubation the hold control showed a virus above 1 log₁₀, however this can be explained by the duration of the experiment. While for the 30 min CVI experiment the hold control was retrieved approximately 150 min (5×30 min) after the spiked test item was prepared, for the 60 min CVI experiment the hold control was retrieved approximately 300 min (5×30 min) after the spiked test item was prepared. Therefore the virus loss observed in the hold control sample can be explained due to the extended exposure to the physico-chemical conditions (pH, salt, buffer, temperature, . . . ) of the spiked test item. Despite the virus loss observed in the HC for the 60 min experiment, it is clear that virus inactivation was due to the contact with the S/D components, as observed at 2 V_{R}, which occurred before 150 min after the spiked test item preparation—the time elapsed for HC sampling in the 30 min CVI experiment.

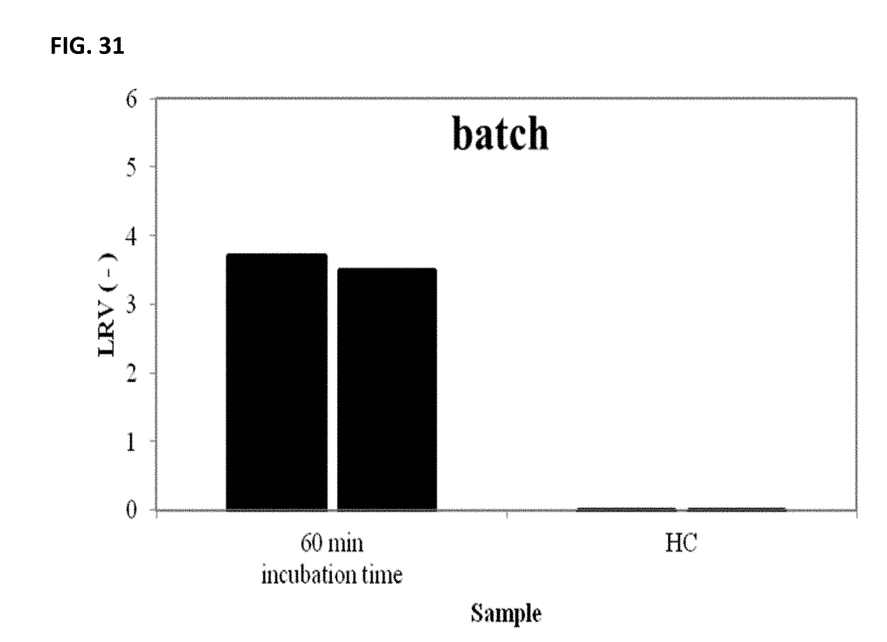

Batch experiments were performed by a person skilled in the art following the industry-relevant guidelines for comparison. The data resulting from the traditional batch (shown in FIG. 31) shows a LRV of 3.5-3.7 at 60 min incubation time, which is comparable with those obtained in the CVIR in the continuous operation mode. This direct comparison shows that the continuous virus inactivation using the CVIR is as effective as the traditional batch operation.

This indicates that continuous virus inactivation according to the invention is highly advantageous, because it is as effective as the ideal inactivation conditions of viral inactivation in the batch mode (e.g. essentially equal residence time for all parts of the mixture due to a narrow residence time distribution, leading to efficient viral inactivation in all parts of the mixture), while providing the additional advantage that it can be carried out continuously.

It will be understood by a person skilled in the art that the conditions used in the viral inactivation example are not limiting to the scope of the invention. For example, while poly(methyl methacrylate) (PMMA) spherical non-porous beads with diameters ranging from 200 to 400 μm with a mean diameter of 300 μm were used as an example, any structure having multiple interconnected channels, for example any column packed with non-porous beads, can be used in accordance with the invention. Similarly, while a cylindrical tube packed using a custom-built vibration-assisted packing station was used as the CVIR having a packed height of 132 mm, a void volume of 10.66±0.06 mL and a CVIR volume of 9.40±0.15 mL, any other CVIR as defined by the present invention can be used.

### INDUSTRIAL APPLICABILITY

The methods, processes and products of the invention are useful for the incubation of substances in industrial manufacturing processes. For example, the invention can be used for the industrial production of biopharmaceuticals. Thus, the invention is industrially applicable.

### REFERENCES

(1) WO 2015 158776 A1

(2) Klutz S, Kurt S K, Lobedann M, Kockmann N. Narrow residence time distribution in tubular reactor concept for Reynolds number range of 10-100. Chem Eng Res Des 2015; 95:22-33.

(3) Klutz S, Lobedann M, Bramsiepe C, Schembecker G. Continuous viral inactivation at low pH value in antibody manufacturing. Chemical Engineering and Processing: Process Intensification 2016; 102:88-101.

(4) WO 2015135844 A1

(5) Kateja N, Agarwal H, Saraswat A, Bhat M, Rathore A S. Continuous precipitation of process related impurities from clarified cell culture supernatant using a novel coiled flow inversion reactor (CFIR). Biotechnology Journal 2016.

(6) EP 3 088 006 A1

(7) Wold, S. Wold, S., Sjostrom, M., Eriksson, L., PLS-regression: a basic tool of chemometrics. Chemometrics and Intelligent Laboratory Systems 2001, 58, 109-130.

(8) Levenspiel, Chemical Reaction Engineering, 3rd ed., John Wiley & Sons, 1999

1. A method for incubating a mixture of at least two liquids, the method comprising:
i) mixing said at least two liquids to obtain a mixture; and
ii) passing said mixture through a structure having multiple interconnected channels, thereby incubating said mixture.

2. The method according to claim 1, wherein the method is a continuous-flow method.

3. The method according to claim 1, wherein said mixing and passing is carried out continuously.

4. The method according to claim 1, wherein the structure having multiple interconnected channels is a packed bed of non-porous beads.

5.-6. (canceled)

7. The method according to claim 4, wherein:
a) the non-porous beads comprise a mean particle diameter in the range of 0.05-1 mm, 0.05-0.6 mm, 0.05 to 0.5 mm, or 0.05-0.3 mm;
b) 95% of the non-porous beads do not deviate from a mean particle diameter by more than 50%, more than 35%, or more than 20%; or
c) both a) and b).

8. (canceled)

9. The method according to claim 1, wherein the structure having multiple interconnected channels has a length of at least 5 cm, or at least 10 cm, or at least 20 cm, or at least 30 cm, or at least 50 cm, or at least 70 cm, or at least 100 cm.

10. (canceled)

11. The method according to any one of claim 4, wherein:
a) the packed bed of non-porous beads is obtained by a method which comprises subjecting said non-porous beads to a vibration treatment
b) the fraction of the volume of voids over the total volume is in the range of 0.2 to 0.45; or
c) both a) and b).

12.-18. (canceled)

19. The method according to claim 1, wherein the method is for virus inactivation, and wherein a first of said at least two liquids is a liquid potentially containing a virus, and wherein a second liquid of said at least two liquids comprises a virus-inactivating agent, and wherein the virus is optionally an enveloped virus.

20. The method according to claim 19, wherein said first liquid comprises a biopharmaceutical drug.

21. -26. (canceled)

27. The method according to claim 19, wherein the method achieves at least a 1 Log10 reduction value (LRV), at least a 2 LRV, at least a 4 LRV or at least a 6 LRV for at least one virus.

28. -29. (canceled)

30. The method according to claim 1 , wherein the Bodenstein number of said mixture when passing through said structure having multiple interconnected channels is equal to or higher than 50, equal to or higher than 300, equal to or higher than 400, equal to or higher than 500, equal to or higher than 600, or equal to or higher than 800.

31. A method for preparing a biopharmaceutical drug, the method comprising performing the method of claim 20 and recovering said biopharmaceutical drug.

32. A device for the preparation of a biopharmaceutical drug, the device comprising a packed bed of non-porous beads, wherein the device comprises at least one of:
a) non-porous beads comprising a mean particle diameter in the range of 0.05-1 mm, 0.05-0.6 mm, 0.05-0.5 mm, or 0.05-0.3 mm;
b) non-porous beads that do not deviate from a mean particle diameter by more than 50%, more than 35%, or more than 20%; or
c) the packed bed of non-porous beads has a length of at least 5 cm, at least 10 cm, at least 20 cm, at least 30 cm, at least 50 cm, at least 70 cm, or at least 100 cm.

33.-38. (canceled)

39. The device according to claim 32, wherein:
a) the packed bed of non-porous beads is obtained by a method which comprises subjecting said non-porous beads to a vibration treatment;
b) the fraction of the volume of voids over the total volume is in the range of 0.2 to 0.45; or
c) both a) and b).

40.-48. (canceled)

49. The device according to claim 32, wherein the device is a continuous-flow reactor.

50. A method for modification of a continuous-flow virus inactivation process, wherein the modification comprises using a structure having multiple interconnected channels for continuous-flow virus inactivation, and passing a mixture of at least two liquids through said structure, thereby incubating said mixture for virus inactivation and wherein said continuous-flow virus inactivation process is optionally a process for the preparation of a biopharmaceutical drug.

51. (canceled)

52. The method according to claim 50, wherein said virus inactivation process uses a virus-inactivating agent for virus inactivation, and wherein a first of said at least two liquids is a liquid potentially containing a virus, and wherein a second liquid of said at least two liquids comprises a virus-inactivating agent, and wherein the virus is optionally an enveloped virus.

53.-55. (canceled)

56. The method according to claim 50, wherein the modification comprises modifying the virus inactivation process to achieve at least a 1 Log10 reduction value (LRV), at least a 2 LRV, at least a 4 LRV or at least a 6 LRV for at least one virus.

57. The method according to claim 50, wherein the modification comprises modifying the virus inactivation process such that Bodenstein number of the mixture passing through said structure having multiple interconnected channels is equal to or higher than 50, equal to or higher than 300, equal to or higher than 400, equal to or higher than 500, equal to or higher than 600, or equal to or higher than 800.

58.-59. (canceled)

60. The method according to claim 56, wherein the modification comprises adjusting the flow through time of said mixture in said structure to achieve said Log10 reduction value (LRV), and wherein the flow through time is adjusted by adjusting the superficial linear velocity of the mixture, the void volume of said structure, or both the superficial linear velocity of the mixture and the void volume.
